(19) United States
(12) Patent Application Publication
(10) Pub. No.: US 2026/0008177 A1
(43) Pub. Date: Jan. 8, 2026

(54) MULTI-TASK AI SYSTEM FOR DYNAMIC AND INTELLIGENT ROBOTIC TASK PLANNING BASED ON USER INPUT

(71) Applicant: INTUITIVE SURGICAL OPERATIONS, INC., Sunnyvale, CA (US)

(72) Inventors: Omid Mohareri, San Fraancisco, CA (US); Muhammad Abdullah Jamal, Sunnyvale, CA (US)

(21) Appl. No.: 19/255,065

(22) Filed: Jun. 30, 2025

Related U.S. Application Data
(60) Provisional application No. 63/667,234, filed on Jul. 3, 2024.

(51) Int. Cl. B25J 9/16; G16H 40/60

(52) U.S. Cl. CPC B25J 9/163; G16H 40/60

(57) ABSTRACT

Systems and methods are described for determining and performing tasks for medical procedures using adaptive artificial intelligence. The system may include one or more repositionable structures configured to support respective instruments, and a control system operably coupled to the repositionable structure, the control system configured to (i) receive a plurality of data streams from one or more data sources, (ii) analyze, according to a task generation machine learning model constitution, the data streams to identify a plurality of tasks to be performed by the one or more repositionable structures; (iii) filter, according to a task selection machine learning model, the identified tasks; (iv) detect a user input and modify, based on the user-input, a data streams, a machine learning model, or operation of a repositionable structure; (v) select a task to be performed; and (vi) control the repositionable structures to perform the selected task.

### CROSS-REFERENCE TO RELATED APPLICATIONS

This application claims priority to and the benefit of the filing date of provisional U.S. Patent Application No. 63/667,234 entitled “MULTI-TASK AI SYSTEM FOR DYNAMIC AND INTELLIGENT ROBOTIC TASK PLANNING BASED ON USER INPUT,” filed on Jul. 3, 2024. The entire contents of the provisional application are hereby expressly incorporated herein by reference.

### TECHNICAL FIELD

The present disclosure relates generally to computer-assisted systems and more particularly to adapting performance of medical procedures performed by computer-assisted systems based on user inputs.

### BACKGROUND

Medical procedures often require various tasks to performed by individuals or devices. Computer-assisted manipulator systems (“manipulator systems”), sometimes referred to as robotically assisted systems or robotic systems, may include one or more medical devices, equipment, and sensors that can be operated with the assistance of an electronic controller (e.g., computer or control system) to move and control functions of one or more instruments or medical devices. A computer-assisted medical system generally includes a robotic system with mechanical links connected by joints. An instrument is removably (or permanently) coupled to one of the links, typically a distal link of the plural links. In some embodiments, the computer-assisted medical systems are used in conjunction with one or more auxiliary devices (e.g., a surgical bed, an insufflator, etc.).

Conventional systems for monitoring medical environments do not provide any functionality relating to efficiently adapting how systems determine what tasks need to be performed for a medical procedure and generate outputs relating to specific tasks to be autonomously performed by manipulator systems. To this end, in some conventional systems, the base model trained to perform this functionality may be fine-tuned to support new capabilities and/or tasks. However, fine-tuning systems to have more additional capabilities typically requires large sample sizes and long set-up times to deploy a system ready for use in a medical setting. As systems and machine learning models become more complex, performing tuning, storing, and providing tuned models requires ever more user input and time, and are impractical to implement in large scales. In other conventional approaches, a frozen pre-trained model is utilized in conjunction with pre-engineered prompt. However, these pre-engineered prompts require significant manual effort to ensure sufficient model performance. Accordingly, this approach has inferior execution of the tasks.

Accordingly, there is a need for improved techniques that enable dynamic adaptation of models and systems to assist in medical procedures while maintaining the task performance capabilities associated with fine-tuned models. Such techniques may allow improved surgical outcomes, improved task workflows, reduced system setup and calibration times, and reduced system training and setup times. These improvements further allow for wider access to surgical treatment and diagnosis across a broad range of medical and clinical domains.

### SUMMARY

The following presents a simplified summary of various examples described herein and is not intended to identify key or critical elements or to delineate the scope of the claims.

In some aspects, the techniques described herein relate to a computer-assisted system for performing automated tasks, the system comprising (a) one or more repositionable structures configured to support respective instruments; and (b) a control system operably coupled to the repositionable structure, wherein the control system is configured to (1) receive a plurality of data streams from one or more data sources; (2) analyze, via a task generation machine learning model, the data streams to identify a plurality of tasks to be performed by the one or more repositionable structures, wherein a task generation constitution is input into the task generation machine learning model to control how the task generation machine learning model analyzes the data streams; (3) filter, via a task selection machine learning model, the identified tasks, wherein a task selection constitution is input into the task selection machine learning model to control how the task selection machine learning model filters the identified tasks; (4) detect a user input indicative of at least (i) one of the plurality of data streams, (ii) one or more machine learning models, or (iii) the operation of the repositionable structures; (5) based on the user input, configure or modify at least one of: (i) which data streams are included in the plurality of data streams; (ii) the task generation constitution to modify how the task generation machine learning model generates tasks, and (iii) an input into a robotics transformer model configured to generate outputs used to control the one or more repositionable structures or instruments; (6) select a task to be performed based on an output of the task selection machine learning model; and (7) control, based on one or more outputs of the robotics transformer model, the repositionable structures to perform the selected task.

In some aspects, the techniques described herein relate to a method for performing automated tasks via a computer-assisted system comprising one or more repositionable structures configured to support respective instruments, and a control system operatively coupled to the one or more repositionable structures, the method comprising (1) receiving a plurality of data streams from one or more data sources; (2) detecting a user input indicative of operation of the repositionable structures; (3) configuring, based on the user input, at least one of (i) which data streams are included in the plurality of data streams, (ii) a task generation constitution configured to modify how the task generation machine learning model generates tasks, and (iii) an input into a robotics transformer model configured to generate outputs used to control of the one or more repositionable structures or instruments; (4) analyzing, via a task generation machine learning model, the data streams to identify a plurality of tasks to be performed by the one or more repositionable structures, wherein the task generation constitution is input into the task generation machine learning model to control how the task generation machine learning model analyzes the data streams; (5) filtering, via a task selection machine learning model, the identified tasks, wherein a task selection constitution is input into the task selection machine learning model to control how the task selection machine learning model filters the identified tasks; (6) selecting a task to be performed based on an output of the task selection machine learning model; and (7) controlling the repositionable structures to perform the selected task.

In some aspects, the techniques described herein relate to a computer-readable media storing instructions that, when executed by a control system of a computer-assisted system, causes the control system to perform any of the methods described herein.

It is to be understood that both the foregoing general description and the following detailed description are illustrative and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTION OF THE DRAWINGS

FIG. 1 is a diagram of a computer-assisted system in accordance with one or more embodiments.

FIG. 2 is a schematic diagram of a system for performing tasks in furtherance of a medical procedure using adaptive artificial intelligence based on user input.

FIG. 3 is a schematic diagram of a robotic action module for generating actions tokens to control a repositionable structure.

FIG. 4 is a schematic diagram of an example task generation module for determining task specific data streams for controlling a repositionable structure.

FIG. 5 is a schematic diagram of an example task selection module for determining task specific data streams for controlling a repositionable structure.

FIG. 6 is a schematic diagram of an example data modality selection module for determining task specific data streams for controlling a repositionable structure.

FIG. 7 is a schematic diagram of an example task assignment module for assigning tasks to perform a procedure.

FIG. 8 is a schematic diagram of an example user input module for detecting user input and modifying one or more data streams and/or constitutions according to the user input.

FIG. 9A is a schematic diagram illustrating a process for detokenizing action tokens.

FIGS. 9B and 9C depict de-tokenized commands for controlling a repositionable structure.

FIG. 10 is a flow diagram of a method for determining and performing tasks for a medical procedure utilizing adaptive artificial intelligence based on user input and/or preferences.

Examples of the present disclosure and their advantages are best understood by referring to the detailed description that follows. It should be appreciated that like reference numerals are used to identify like elements illustrated in one or more of the figures, wherein showings therein are for purposes of illustrating examples of the present disclosure and not for purposes of limiting the same.

### DETAILED DESCRIPTION

In this description, specific details are set forth describing some embodiments consistent with the present disclosure. Numerous specific details are set forth in order to provide a thorough understanding of the embodiments. It will be apparent, however, to one skilled in the art that some embodiments may be practiced without some or all of these specific details. The specific embodiments disclosed herein are meant to be illustrative but not limiting. One skilled in the art may realize other elements that, although not specifically described here, are within the scope and the spirit of this disclosure. In addition, to avoid unnecessary repetition, one or more features shown and described in association with one embodiment may be incorporated into other embodiments unless specifically described otherwise or if the one or more features would make an embodiment non-functional.

Further, the terminology in this description is not intended to limit the invention. For example, spatially relative terms-such as “beneath”, “below”, “lower”, “above”, “upper”, “proximal”, “distal”, and the like-may be used to describe the relation of one element or feature to another element or feature as illustrated in the figures. These spatially relative terms are intended to encompass different positions (i.e., locations) and orientations (i.e., rotational placements) of the elements or their operation in addition to the position and orientation shown in the figures. For example, if the content of one of the figures is turned over, elements described as “below” or “beneath” other elements or features would then be “above” or “over” the other elements or features. A device may be otherwise oriented and the spatially relative descriptors used herein interpreted accordingly. Likewise, descriptions of movement along and around various axes include various special element positions and orientations. In addition, the singular forms “a”, “an”, and “the” are intended to include the plural forms as well, unless the context indicates otherwise. Additionally, the terms “comprises”, “comprising”, “includes”, and the like specify the presence of stated features, steps, operations, elements, and/or components but do not preclude the presence or addition of one or more other features, steps, operations, elements, components, and/or groups. Components described as coupled may be electrically or mechanically directly coupled, or they may be indirectly coupled via one or more intermediate components.

Elements described in detail with reference to one embodiment, implementation, system, or module may, whenever practical, be included in other embodiments, implementations, systems, or modules in which they are not specifically shown or described. For example, if an element is described in detail with reference to one embodiment and is not described with reference to a second embodiment, the element may nevertheless be claimed as included in the second embodiment. Thus, to avoid unnecessary repetition in the following description, one or more elements shown and described in association with one embodiment, implementation, or application may be incorporated into other embodiments, implementations, or aspects unless specifically described otherwise, unless the one or more elements would make an embodiment or implementation non-functional, or unless two or more of the elements provide conflicting functions.

In some instances, well known methods, procedures, components, and circuits have not been described in detail so as not to unnecessarily obscure aspects of the embodiments.

This disclosure describes various devices, elements, and portions of computer-assisted systems and elements in terms of their state in three-dimensional space. As used herein, the term “position” refers to the location of an element or a portion of an element (e.g., three degrees of translational freedom in a three-dimensional space, such as along Cartesian x-, y-, and z-coordinates). As used herein, the term “orientation” refers to the rotational placement of an element or a portion of an element (e.g., three degrees of rotational freedom in three-dimensional space, such as about roll, pitch, and yaw axes, represented in angle-axis, rotation matrix, quaternion representation, and/or the like). As used herein, and for a device with a kinematic series, such as with a repositionable structure with a plurality of links coupled by one or more joints, the term “proximal” refers to a direction toward a base of the kinematic series, and “distal” refers to a direction away from the base along the kinematic series.

As used herein, the term “pose” refers to the multi-degree of freedom (DOF) spatial position and orientation of a coordinate system of interest attached to a rigid body. In general, a pose includes a pose variable for each of the DOFs in the pose. For example, a full 6-DOF pose for a rigid body in three-dimensional space would include 6 pose variables corresponding to the 3 positional DOFs (e.g., x, y, and z) and the 3 orientational DOFs (e.g., roll, pitch, and yaw). A 3-DOF position only pose would include only pose variables for the 3 positional DOFs. Similarly, a 3-DOF orientation only pose would include only pose variables for the 3 rotational DOFs. Further, a velocity of the pose captures the change in pose over time (e.g., a first derivative of the pose). For a full 6-DOF pose of a rigid body in three-dimensional space, the velocity would include 3 translational velocities and 3 rotational velocities. Poses with other numbers of DOFs would have a corresponding number of velocities translational and/or rotational velocities.

This disclosure occasionally refers to the disclosed techniques being applied to “patients” undergoing a “medical procedure.” It should be appreciated that these references are not intended to limit the application of the disclosed techniques to applied medicine contexts. For example, the described techniques can be applied to facilitate physician training, equipment testing and/or calibration, and/or other contexts. Accordingly, any reference to the term “patient” is done for ease of explanation and also envisions the application of the described techniques to a generic “subject.”

The word “task” is used herein to refer to a discrete portion of procedure that may be autonomously, semi-autonomously, or manually implemented in furtherance of a procedure. For example, a task may be to move an endoscope to a particular portion, to advance an instrument to a particular depth, to replace an instrument coupled to a manipulator, and so on. In some embodiments, a task is associated with component tasks to accomplish an overall goal. For example, a task to analyze a worksite may include component tasks related to moving an endoscope to view the worksite, advancing an instrument to predetermined depth, and enabling a functionality supported by the instrument.

Aspects of this disclosure are described in reference to computer-assisted systems, which can include devices that are teleoperated, externally manipulated, autonomous, semiautonomous, and/or the like. Further, aspects of this disclosure are described in terms of an implementation using a teleoperated surgical system, such as the da Vinci® Surgical System commercialized by Intuitive Surgical, Inc. of Sunnyvale, California. Knowledgeable persons will understand, however, that inventive aspects disclosed herein may be embodied and implemented in various ways, including teleoperated and non-teleoperated, and medical and non-medical embodiments and implementations. Implementations on da Vinci® Surgical Systems are merely exemplary and are not to be considered as limiting the scope of the inventive aspects disclosed herein. For example, techniques described with reference to surgical instruments and surgical methods may be used in other contexts. Thus, the instruments, systems, and methods described herein may be used for humans, animals, portions of human or animal anatomy, industrial systems, general robotic, or teleoperated systems. As further examples, the instruments, systems, and methods described herein may be used for non-medical purposes including industrial uses, general robotic uses, sensing or manipulating non-tissue work pieces, cosmetic improvements, imaging of human or animal anatomy, gathering data from human or animal anatomy, setting up or taking down systems, training medical or non-medical personnel, and/or the like. Additional example applications include use for procedures on tissue removed from human or animal anatomies (with or without return to a human or animal anatomy) and for procedures on human or animal cadavers. Further, these techniques can also be used for medical treatment or diagnosis procedures that include, or do not include, surgical aspects.

The disclosure generally relates to systems and methods for training and intelligently providing adaptive artificial intelligence (AI) for performing and assisting in operating room tasks. While the disclosure primarily focuses on adapting constitutions that control task generation, task filtering, data modality selection, task assignment, and robotic transformer machine learning models, the adaptions can be made to any set of inputs to a machine learning model implemented in a control system. In some embodiments, the control system disclosed is configured to detect a user input indicative of an intention to change operation of the control system. The control may include a user input processing module to identify which machine learning models should be adapted in view of the user input. Accordingly, techniques described herein relate to routing the user input and/or processed data derived from the user input to adapt the constitution that controls an aspect of the autonomous operation indicated by the user input. As a result, the performance of the machine learning models can be adapted without the cost and time of tuning the underlying models.

To adapt a constitution, in some embodiments the systems may store sets of predefined rules that can be incorporated into a constitution. For example, the sets of predefined rules may relate to how to prioritize different types of tasks (e.g., should speed of procedure be prioritized, should manual guidance be prioritized, etc.). Accordingly, the systems may be configured to process user input to identify an appropriate set of rules and update a constitution in accordance therewith. In some embodiments, if the systems do not detect an appropriate set of rules, the systems may utilize a generative AI model to generate one or more rules intended to implement the user input.

The user input may be provided in natural language (e.g., via a chatbot-type interface), as audio data, as non-linguistic user interactions with a GUI (e.g., tapping or gesturing on a touch-sensitive display screen), or any other suitable method of determining user intended operation of a robotic system for performing medical operations. By modifying the constitutions based on user inputs, the systems are able to dynamically optimize the outputs of a given model for a specific task at hand, environment, and/or conditions without having to fine-tune the models and systems. As a result, automated control of the robotic system more closely aligns with user indications while still preserving strong task performance capabilities.

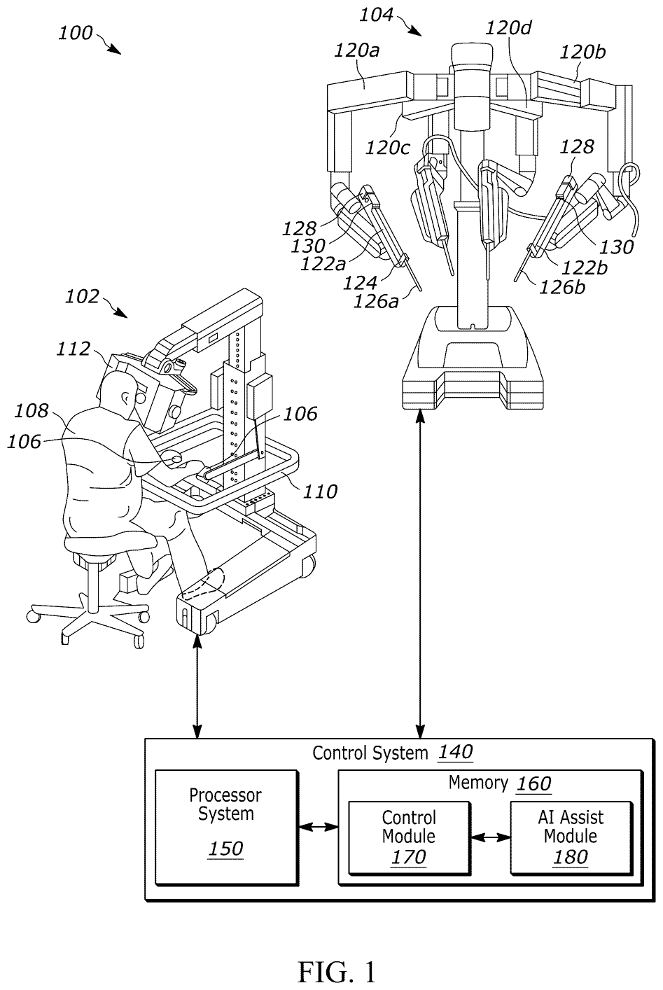

FIG. 1 is a simplified diagram of an example computer-assisted system 100, according to various embodiments. The computer-assisted system 100 may be a computer-assisted medical system for assisting with performing tasks for medical procedures. Further, the computer-assisted system 100 may utilize adaptable AI models for determining and performing medical tasks as described herein. The computer-assisted system 100 may further determine and utilize task-specific data streams for performing one or more medical tasks, and assign tasks as described herein. In some examples, the computer-assisted system 100 is a teleoperated system. In medical examples, the computer-assisted system 100 can be a teleoperated medical system such as a surgical system. As shown, the computer-assisted system 100 includes a follower device 104 that can be teleoperated by being controlled by one or more leader devices (also called “leader input devices” when designed to accept external input), described in greater detail below. Systems that include a leader device and a follower device are referred to as leader-follower systems, and also sometimes referred to as master-slave systems. Also shown in FIG. 1 is an input system that includes a workstation 102 (e.g., a console), and in various embodiments the input system can be in any appropriate form and may or may not include the workstation 102.

In the example of FIG. 1, the workstation 102 includes one or more leader input devices 106 that are designed to be contacted and manipulated by an operator 108. For example, the workstation 102 may comprise one or more leader input devices 106 for use by the hands, the head, or some other body part(s) of operator 108. The leader input devices 106 in this example are supported by the workstation 102 and can be mechanically grounded. In some embodiments, an ergonomic support 110 (e.g., forearm rest) can be provided on which the operator 108 can rest his or her forearms. In some examples, the operator 108 can perform tasks at a worksite within a workspace near the follower device 104 during a procedure, by commanding the follower device 104 using the leader input devices 106. In a medical example, the worksite may be a surgical worksite associated with a patient.

A display device 112 is also included in the workstation 102. The display device 112 may be configured to display images for viewing by the operator 108. The display device 112 can be moved in various DOFs to accommodate the viewing position of the operator 108 and/or to provide control functions. In embodiments where the display device 112 provides control functions, the leader input devices 106 may include the display device 112. In the example of the computer-assisted system 100, displayed images may depict a worksite at which the operator 108 is performing various tasks by manipulating the leader input devices 106 and/or the display device 112. In some examples, images displayed by display device 112 may be received by the workstation 102 from one or more imaging devices arranged at a worksite. In other examples, the images displayed by the display device 112 may be generated by the display device 112 (or by a different connected device or system), such as for virtual representations of tools, the worksite, or for user interface components. As will be explained below, in some embodiments the display device 112 may display one or more tasks for the operator 108 to perform with respect to any component of the computer-assisted system 100.

In examples, the display device 112 may be a touch-screen device and may receive user-input via the touch screen. The touch screen may provide a user interface that a user may select from various options to provide one or more user preferences for performing a procedure or medical task. Additionally, the display device 112 may display one or more images such as an intraoperative or pre-operative image of a patient, organ, surgical site etc., and the user may provide user input via the touch screen to indicate one or more regions or elements displayed in the intraoperative or pre-operative images. The display device 112 may provide one or more images or user interfaces and a user may interact with and provide user indications and input via another device such as a keyboard, mouse, audio device, etc. For example, a user may provide text input such as natural language text via a keyboard to provide a user input to the system. A user may use a mouse or another similar device to click on or indicate selection of an option or user feedback based on one or more images presented by the display device. The workstation 102 may further include a microphone that may capture and record audio of a user providing indications of user preferences and user inputs to the system. One or more processors, or controllers as discussed further herein, may then derive natural language text from the recorded audio data to derive a user input to the system 100. In examples, the user input may further be determined from the audio data as a user responding to one or more prompts to confirm, reject, or otherwise indicate a user preference or input. Additionally, user input may be provided via one or more sources such as video, haptic input, data banks, instruments (e.g., by a user changing a setting on an instrument or auxiliary device), sensors, etc. It should be appreciated that while the foregoing describes obtaining the user input from the display device 112 of the workstation 102, in other embodiments, the user input may be obtained by other display devices associated with the operating room that are interacted with by personnel other than the operator 108.

As illustrated, the computer-assisted system 100 also includes a follower device 104 that can be commanded by the workstation 102. In a medical example, the follower device 104 can be located near an operating table (e.g., a table, bed, or other support) on which a patient can be positioned. In some medical examples, the workspace is provided on an operating table, e.g., on or in a patient, simulated patient, or model, training dummy, etc. (not shown). As illustrated, the follower device 104 may include a plurality of repositionable structures 120 (sometimes referred to as “manipulator arms” in robotic embodiments). In some embodiments, the repositionable structures 120 may include a plurality of links that are rigid members and joints that can be individually actuated as part of a kinematic series. Additionally, each of the repositionable structures 120 is configured to couple to an instrument 122. While FIG. 1 illustrates a follower device 104 that has four repositionable structures 120a-120d, in other embodiments, the follower device 104 may include one, two, three, four, five, six, or additional or fewer repositionable structures 120a-120d.

The instrument 122 can include, for example, a working portion 126 and one or more structures for supporting and/or driving the working portion 126. Example working portions 126 include end effectors that physically contact or manipulate material, energy application elements that apply electrical, RF, ultrasonic, or other types of energy, sensors that detect characteristics of the workspace environment (such as temperature sensors, imaging devices, etc.), and the like. In various embodiments, examples of instruments 122 include, without limitation, a sealing instrument, a cutting instrument, a sealing-and-cutting instrument, an energy instrument for applying energy, a gripping instrument (e.g., clamps, jaws), a stapler, an imaging instrument such as one using optical, RF, or ultrasonic imaging modalities, a sensing instrument, an irrigation instrument, a suction instrument, and/or the like. In addition, the instrument 122 may include a transmission mechanism 128 that can be coupled to a drive assembly 130 of the respective repositionable structure 120a-120d. The drive assembly 130 may include a drive and/or other mechanisms controllable from workstation 102 that transmit forces to the transmission mechanism 128 to articular or otherwise actuate the instrument 122.

As illustrated, each instrument 122 may be mounted to a portion of a respective repositionable structure 120a-120d. In FIG. 1, this is shown with the drive assembly 130 physically coupled to the transmission mechanism 128. The distal portion of each repositionable structure 120a-120d further includes a cannula mount 124 to which a cannula (not shown) is mounted. When a cannula is mounted to the cannula mount 124, a shaft of the instrument 122 passes through the cannula and into a workspace.

In various embodiments, one or more of the working portions 126 of the instruments 122 may include an imaging device for capturing images. The imaging device may include any sensing technology capable of acquiring an image. Example imaging instruments include an optical endoscope, a hyperspectral camera, an ultrasonic sensor, etc. Imaging instruments may comprise monoscopic imagers, stereoscopic imagers, and/or the like. Imaging devices based on radiofrequency domains may capture images in any frequency spectrum, including visible light, infrared light, ultraviolet light, and/or the like. The imaging device may include an illumination source to light the region being imaged. In embodiments where the working portions 126 of one or more of the instruments 122 include an imaging device, the instrument 122 may be configured to capture images of a portion of the workspace for display via the display device 112.

In some embodiments, the repositionable structures 120a-120d and/or instruments 122 can be controlled to move the working portion 126 in response to manipulation of the leader input devices 106 by the operator 108. Accordingly, the repositionable structures 120a-120d and/or instruments 122 may be said to “follow” the leader input devices 106 through teleoperation. This enables the operator 108 to perform tasks at the worksite using the repositionable structures 120a-120d and/or instruments 122. For a surgical example, the operator 108 can direct the repositionable structures 120a-120d of the follower device 104 to move the working portions 126 as part of a surgical procedure performed at an internal surgical site that is entered via one or more minimally invasive apertures or natural orifices. It should be appreciated that, in some embodiments, the follower device 104 may include non-teleoperated components that the operator 108 or other medical professional must manually manipulate to a desired pose.

In some embodiments, a repositionable structure 120a of the computer-assisted system 100 may be configured to support a working portion 126a that includes an imaging device (also referred to herein as an “imaging device 126a”). For convenience, an instrument 122 that includes an imaging device is also referred to as an “imaging instrument” herein. The control system 140 may be configured to command the repositionable structure 120a and/or the imaging instrument 122 comprising the imaging device 126a to automatically position and/or orient (“pose”) the field of view (FOV) of the imaging device 126a to provide images of the workspace and/or other instruments 122.

In the illustrated embodiment, a control system 140 is communicatively coupled to the workstation 102. In other embodiments, the control system 140 may be provided as a component of the workstation 102 and/or the follower device 104. During teleoperation, as the operator 108 moves the leader input device(s) 106, one or more sensors configured to detect the leader input device(s) 106 generate spatial and/or orientation movement data that is provided to control system 140. The control system 140 may interpret the spatial and/or orientation information to determine and/or provide control signals to the follower device 104 to control the movement of repositionable structures 120a-120d, instruments 122, and/or working portions 126. In addition to the components of the follower device 104, in some embodiments, the control system 140 is configured to interpret inputs received from the workstation 102 to control operation of one or more auxiliary devices (not depicted) utilized in a procedure. For example, the workstation 102 may be used to control a pose of a surgical bed or operation of an insufflator.

In one embodiment, the control system 140 supports one or more wired communication protocols, (e.g., Ethernet, USB, and/or the like) and/or one or more wireless communication protocols (e.g., Bluetooth, IrDA, HomeRF, IEEE 1102.11, DECT, Wireless Telemetry, and/or the like) for communications between the control system 140 and the workstation 102 and/or the follower device 104.

In some embodiments, the control system 140 may be implemented at one or more computing systems. For example, one or more computing systems may be used to control the follower device 104. As another example, one or more computing systems may be used to control components of the workstation 102, such as movement of a display device 112.

As illustrated, the control system 140 includes a processor system 150, a memory 160, and an artificial intelligent (AI) assist module 180. The memory 160 may store a control module 170. The processor system 150 may include one or more processors having different processing architectures for processing instructions. For example, the one or more processors may be one or more cores or micro-cores of a multi-core processor, a central processing unit (CPU), a microprocessor, a field-programmable gate array (FPGA), an application-specific integrated circuit (ASIC), a digital signal processor (DSP), a graphics processing unit (GPU), a tensor processing unit (TPU), and/or the like.

In some embodiments, the processor system 150 includes circuitry to support one or more communication interfaces (e.g., Bluetooth interface, infrared interface, network interface, optical interface, etc.). Additionally, a communication interface of control system 140 may include an integrated circuit for connecting the control system 140 to a network (not shown) (e.g., a local area network (LAN), a wide area network (WAN) such as the Internet, mobile network, or any other type of network) and/or to another device, such as the workstation 102 and/or the follower device 104.

Additionally, the memory 160 may include non-persistent storage (e.g., volatile memory, such as random access memory (RAM), cache memory), persistent storage (e.g., a hard disk, an optical drive such as a compact disk (CD) drive or digital versatile disk (DVD) drive, a flash memory, a floppy disk, a flexible disk, a magnetic tape, any other magnetic medium, any other optical medium, programmable read-only memory (PROM), an erasable programmable read-only memory (EPROM), a FLASH-EPROM, and/or any other memory chip or cartridge. The non-persistent storage and persistent storage are examples of non-transitory, tangible machine-readable media that can store executable code that, when run by one or more processors (e.g., processor system 150), can cause the one or more processors to perform one or more of the techniques and/or methods disclosed herein.

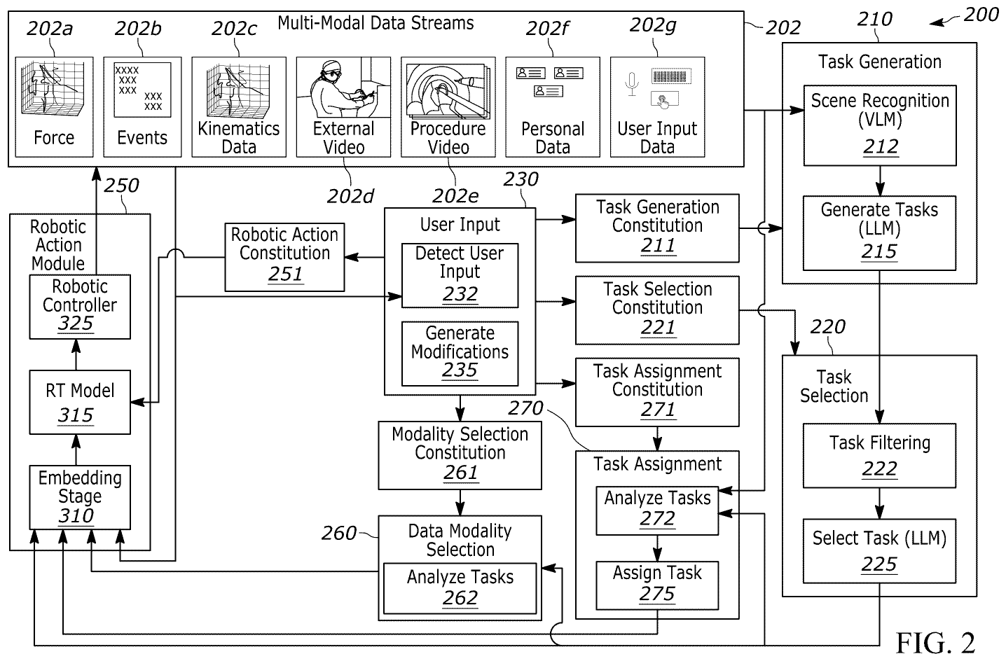

The AI assist module 180 may implement one or more machine learning models and/or training protocols therefore. For example, the AI assist module 180 may implement one or more neural networks, deep learning models, decision trees, support vector machines, linear regression, generative AI models, reinforced learning models, random forests, Naïve Bayes models, large language models (LLMs), generative adversarial networks, foundation models, image recognition models, linear discriminant analysis models, creative applications, autoregressive models, supervised or unsupervised learning models, multimodal models, vision language models (VLMs), vision foundation models (VFMs), large multi-modal models (LMMs), Transformer models (including Robotic Transformer models), or another machine learning or AI model for performing the methods described herein. The structure of the one or more machine learning is described in more detail with respect to FIGS. 2-9C. The AI assist module 180 may include dedicated processors and memory for storing and performing AI processes, or the AI assist module 180 may utilize resources of the processor system 150 and the memory 160 to store and/or perform any processing or tasks required to perform the methods described herein.

Additionally, the control system 140 may also include one or more input devices (such as a touchscreen, keyboard, mouse, microphone, touchpad, electronic pen, or any other type of input device) and/or output devices (such as a display device, a speaker, external storage, a printer, or any other output device). In some embodiments, the control system 140 may be implemented on a particular node of a distributed computing system (e.g., a cloud computing system). As another example, different functionalities associated with the control system 140 may be implemented on different nodes of the distributed computing system. Further, one or more elements of the aforementioned control system 140 may be located at a remote location and connected to the other elements over a network.

In an endoscopic surgery example, the imaging instrument comprising the imaging device 126a may be inserted into the patient prior to the other instruments 122, including a second instrument 122b comprising a second working portion 126b. The second instrument 122b can include any appropriate working portion 126b, and can even include a second imaging device. Accordingly, the imaging device 126a may be maneuvered to positioned to identify a target to which other instruments may interact with as part of another task. The control system 140 may, for example, automatically command the corresponding repositionable structures 120a and 120b to position respective instruments 122a and 122b to perform one or more tasks in tandem, or sequentially based on the specific task, instruments, and positions of the repositionable structures 120a and 120b. In examples, the control system 140 may perform AI processes and algorithms via the AI assist module 180 to identify tasks for performing a medical procedure, and to further assign the tasks to respective personnel for performing the tasks. Additionally, the control system 140 may identify certain tasks to be performed automatically by one or more robotic systems and/or devices, semi-automatically with user assistance or input to the system 100, and tasks that are to be performed entirely manually by personnel. It should be appreciated that some manual tasks may be performed independent of the computer-assisted system 100. For example, some tasks generated by the control system 140 may include sterilizing instruments or a worksite, obtaining an instrument for use later in a procedure, adjusting a pose of the subject, and so on.

The disclosed techniques enable the control system 140 to perform real-time operations (e.g., within 5 ms, within 10 ms, within 20 ms, etc.). Accordingly, the control system 140 is able to identify and control repositionable structures to perform tasks for a medical procedure in a variety of scenarios using adaptable AI. Such techniques improve the efficiency of operating the computer-assisted system or instrument, simplify user control of the computer-assisted system 100, improve the efficiency of the medical procedure by streamlining task workflow, and allow for computer-assisted systems to be used in a much broader range of environments, and under various specific conditions without the needed for additional ML training and/or tuning. Further, although a surgical example is shown, the disclosed techniques provide an improvement to the computer-assisted system 100 in the non-surgical aspects of the procedure, and can be used to improve computer-assisted systems applied in non-medical contexts.

FIG. 2 is a schematic diagram of a system 200 for using adaptive AI for controlling repositionable structures to perform tasks in furtherance of a medical procedure. The tasks may be performed manually, semi-automatically with user input or guidance, or entirely automatically by a robotic structure or system (such as the repositionable structure 120). The system 200 includes a plurality of modules that may be executed by the control system 140 (e.g., via the AI assist module 180 of FIG. 1) to determine and perform the one or more tasks associated with a medical procedure. The system 200 includes sources of multi-modal data that form one or more multi-modal data streams 202, a task generation module 210, a task selection module 220, a user input module 230, a data modality select module 260, a task assignment module 270, and a robotic action module 250. It should be appreciated that in other embodiments, additional or fewer modules may be implemented. Further, in other embodiments, one or more of the described modules may be combined into a single module.

The system 200 includes one or more sources of multi-modal data 202. For example, the multimodal data 202 may include a data stream 202a indicative of force exerted upon an instrument, a data stream 202b indicative of system events generated by the repositionable structure and/or a control system thereof, a data stream 202c indicative of kinematic data associated with the repositionable structure and/or instruments or auxiliary devices associated therewith, an external video data stream 202d, a procedure video data stream 202e (such as image data generated by an endoscope), a data stream 202f indicative of personnel and data associated with personnel that may be available for performing the procedure, a data stream 202g indicative of user input data such as via a user interface, and/or other sources of data that indicate a state of a procedure facilitated by the repositionable structures. Additionally, the data streams 202 may include data retrieved from one or more data based such as preoperative data (e.g., preoperative images, video, patient information, etc.). In examples, the data streams 202a-202g may include system data (e.g., data associated with system events, system capabilities, etc.), and data associated with a medical environment (e.g., via video data, image data, available instruments, etc.). In examples, the multimodal data 202 may include one or more data streams that provide layouts of medical environments or rooms and/or information associated with one or more medical systems and instruments that may be available for a procedure.

The system 200 may be configured to route the multimodal data 202 to a task generation module 210 configured to output one or more tasks that may be performed in furtherance of the procedure based on a procedure state represented by the input multimodal data 202. The task generation module 210 may identify tasks that are to be perform in the near term (e.g., tasks that respond to conditions detected in an input set of multi-modal data) and/or tasks that are to be performed in the long term (e.g., tasks that will need to be performed later in the procedure after one or more near-term tasks are performed). The task generation module 210 may further generate tasks to be performed in the future for a medical procedure scheduled to be performed at a later time or date. By also generating long-term tasks, the system 200 is able to track preparedness for performing the long-term task and generate additional tasks to prepare the control system for performing the long-term task as the procedure advances closer to the appropriate time for performing the long-term tasks. In some embodiments, the task generation module 210 may include component models to facilitate the analysis. For example, the task generation module 210 may include a scene recognition model 212 to parse image data to generate natural language descriptions of the scene represented by the image data (and the corresponding locations in the image data) and a task generation model 215 configured to actually generate the one or more tasks. As one example, the scene recognition model 212 may detect and identify objects in a medical environment such as an operating room (OR) to generate an inventory of objects in the OR.

The task generation module 210 may analyze the multimodal data 202 to identify the tasks to be performed according to a set of rules. For example, a task generation constitution 211 may be input into the task generation module 210 as part of a prompt that also includes at least a portion of the multimodal data streams 202.

In some embodiments, a data stream input into the task generation module 210 may include an embedding indicative of the medical procedure, or of one or more phases of the medical procedure. The system 200 may then add or edit rules of the task generation constitution 211 according to the specific medical procedure or phases of the medical procedure that is to be performed. One such technique for detecting the type of medical procedure and/or phase thereof is described in U.S. Provisional Application No. 63/663,996, the entire disclosure of which is hereby incorporated by reference. Accordingly, in these embodiments, the phase information may be input into the task generation model 215 to generate tasks based on the particular phase for the medical procedure and according to the rules in the task generation constitution 211.

The system 200 may then provide the output tasks (e.g., natural language descriptions of an action to be performed) to a task selection module 220 to select which tasks should actually be implemented by the control system, semi-automatically via user control/feedback and the control system, and/or by an operator thereof. As illustrated, the task selection module may include a task filter model 222 configured to filter the tasks generated by the task generation model 215 and a task selection model 225 configured to select a one or more valid tasks to be performed by the control system and/or an operator thereof.

More particularly, the task selection module 220 may input a task selection constitution 221 input into a task selection model 225 along with one or more input tasks to select which tasks are to be performed. The task selection constitution 221 may include one or more sets of rules that control how the task assignment module 220 filters and selects the identified tasks. In examples, the rules of the task selection constitution 221 may cause the task selection module 220 to filter the various tasks according to one or more available data streams, available personnel, user inputs and preferences, device models, available instruments etc. The task selection module 220 may then select the tasks according to the rules of the task selection constitution 221, and provide the selected tasks to the data modality selection module 260, the task assignment module 270, and/or the robotic action module 250.

The data modality selection module 260 may be configured to analyze the one or more selected tasks to determine one or more task specific data streams needed to implement the selected tasks. To this end, each task of a procedure or phase of a procedure may require a particular type of data to safely and effectively execute the task. Accordingly, the data modality selection module 260 may input a modality selection constitution 261 into a task analysis model 262 along with the selected tasks to determine one or more task specific data streams. Thus, the modality selection constitution 261 may be configured to control how the data modality selection module 260 determines the set of task-specific data streams.

It should be appreciated that a required data modality may include a particular data stream of the multimodal data 202 (e.g., kinematic data or procedure video) or a particular state of the multimodal data (e.g., that the procedure video is required to captured image data of a particular object, such a target anatomy). If a required data stream is unavailable, the rules of the modality selection constitution 261 may instruct the data modality selection module 260 to interact with the task selection module 220 to select a different task where the required data streams are all available and/or the task generation module 210 to generate a task to make the required data stream available (e.g., by enabling a sensor or by commanding a camera to reposition a field of view). The data modality selection module 260 may then provide a selection of the available data streams to the robotic action module 250.

Because the system 200 may be configured to process multiple tasks synchronously, the data modality selection module 260 may be configured to associate the selected available data streams with each task prior to providing the selection to the robotic action module 250. As a result, the robotic action module 250 may be able switch data modalities based on the particular tasks being converted into robotic action.

The task assignment module 270 may be configured to analyze the one or more selected tasks to assign the tasks to respective instruments, end effectors, auxiliary devices, personnel etc., according to rules of a task assignment constitution 271. Additionally, one or more data streams of the plurality of data streams 202 may be input into the task assignment module 270 to assist in the task assignment analysis. While the task assignment module 270 is generally described with respect to instrument assignment, any such description is done for brevity, and envisions similar techniques related to selection of any actor to perform a particular task. The task assignment module 270 includes a task analysis model 272 configured to analyze to identify the which instruments (including combinations of instruments) are capable of implementing the selected tasks and a task assignment model 275 configured to assign the task to respective in. The task analysis model 272 may identify different requirements for each task (e.g., personnel skillsets and qualifications, instrument and system capabilities, etc.). In some embodiments, a particular task may require multiple instruments and/or personnel to perform different aspects of a given task. Accordingly, the task analysis model 272 may be configured to divide the task into subtasks that are assigned to different instruments and/or personnel. The task analysis model 272 may analyze and identify instruments and/or personnel, and identify the different requirements for the tasks according to, for example, one or more rule sets included in the task assignment constitution 271.

The task assignment module 270 further includes a task assignment module 275 (such as an LLM) that analyzes the requirements for the input tasks (and/or subtasks thereof) and assigns the various tasks to appropriate instrument according to one or more rule sets of the task assignment constitution 271. That is, the task assignment module 270 may be configured to input both the selected tasks (and/or subtasks thereof) and the task assignment constitution 271 to the task assignment module 275 to determine the appropriate task assignments. It should be appreciated that if required instruments are not present or available, the rules of the task assignment constitution 271 may instruct the task assignment module 270 to send an indication to the task generation module 210 and/or the task selection module 220 to generate and/or select alternative tasks that can be performed based on the current instrument availability.

To perform semi-autonomous and autonomous tasks via the assigned instruments, the system 200 includes the robotic action module 250. As illustrated, the robotic action module 250 may be configured to receive the selected tasks from the task selection module 220, task assignments from the task assignment module 270, task-specific data streams from the data modality module 260, and/or the multimodal data 202. The robotic action module 250 may then process the multimodal data 202 (e.g., by embedding the determined task-specific data streams via the embedding stage 310), and the selected tasks to generate action tokens to control the manipulator system to perform the selected tasks. The robotic action module 250 may input the embeddings of the various inputs, and a robotic action constitution 251 into a RT model 315 to generate one or more action tokens. The action tokens may be natural language descriptions of a robotic action to be performed by the manipulator system in furtherance of a selected task. The robotic action module 250 may then convert the action tokens to command one or more components of a computer-assisted robotic device (such as the follower device 104 of FIG. 1) to perform one or more of the selected tasks.

It should be appreciated that in some embodiments, one or more of the modules 210, 220, 230, 260, and 270 may be combined into a single module, for example, by implementing chain of thought (CoT) or other recurrent prompting techniques such that a single prompt performs the described functionality corresponding to the individual modules. Additionally, in other embodiments, the system 200 may include additional modules that generate inputs to the robotic action module 250 to implement autonomous tasks.

Generally, the constitutions of the system 200 (e.g., the constitutions 211, 221, 231, 261, 271) may include various sets of rules according to different types or categories of rules. For example, the constitutions may include foundational rules that define allowable robotic actions (e.g., limitations on instrument operation for patient and user safety, such as Asimov's three rules), safety rules that define safe operation of the computer-assisted medical system (e.g., spatial limitations, sanitization standards, etc.), embodiment rules that define capabilities and limitations of the computer-assisted medical system, associated instruments and devices (e.g., degrees of freedom of robotic arm movement, imaging capabilities of endoscopes and imaging devices, volume capabilities of extraction and injection devices, etc.), and/or user preference rules (e.g., rules defined and input by a user or personnel for performing a procedure or task).

As described herein, the system 200 may be configured to adapt the constitutions of the system 200 based on one or more user inputs. Accordingly, as illustrated, the system 200 also includes a user input module 230 configured to receive one or more user inputs and/or instructions from a user to adapt the constitutions in view thereof. Accordingly, the user input module 230 includes a user input detection model 232 via which the system 200 detects the user input. For example, a user may interact with a user interface (e.g., a chatbot interface, a intraoperative imaging interface, etc.) to provide the user inputs and/or instructions. In this example, the user input detection model 232 may detect the text entered into user interface. As another example, the user input detection model 230 may receive a data stream 202g indicative of the user input or instructions. In this example, one or more sensors or devices in an environment, of an instrument or auxiliary device, etc. may detect the user input (e.g., via an audio sensor or a video sensor) and process the data (e.g., by transcribing and/or generating a label indicative of a gesture) using the user input detection model 232.

The user input module 230 may then analyzes the user input using a modification generation model 235 to modify operation of the system 200. As one example, a user may provide an instruction to begin a certain phase of a procedure. In response, the system 200 may update the task generation constitution 211 (and/or other constitutions described herein) to include procedure rules associated with the new phase of the procedure. To this end, because the task generation model 215 has a limited context window, swapping in the procedure rules for the particular phase of the procedure enables the task generation constitution 211 to be a smaller size, thereby enabling faster processing of the prompt of the ability to consider additional inputs to generate tasks more tailored to the current situation.

Similarly, the user input may indicate (or confirm an automated determination based on event data) that a particular instrument and/or end effector has been coupled to the manipulator system. In response, the system may modify the embodiment rules to include rules specific to the new instruments/end effectors and remove rules related to instruments/end effectors de-coupled from the manipulator system. Again, this enables the constitutions described herein to include rules more tailored to current situation, thereby reducing the size of the constitution.

As another example, the user may indicate a particular region in the image data associated with the subject's anatomy that should be avoided. In response, the system 200 may label the region and insert the label into a safety rule template included in the constitutions. As a result, the tasks output by the task generation model 210 and/or selected by the task selection module 220 may be specifically generated to avoid the labeled region.

As yet another example, the user may indicate a set of user preferences. For example, the user may prefer to certain orders of tasks, certain positioning of the end effectors prior to interacting with a worksite, etc. In some embodiments, the user may specify these preferences to the system 200 prior to or during the procedure. Additionally or alternatively, the user may have configured their own set of rules in advance of the procedure. In these embodiments, the user input may identify the user such that the system 200 is able to obtain the corresponding set of rules. Regardless, after identifying the user preferences, the system 200 may update user preference rules to include the identified rules.

While the foregoing describes various changes to the rules included the disclosed constitutions, in other embodiments, other changes can be provided. For example, a user may instruct the system to change a state of the procedure video data stream 202e to include a particular view. Accordingly, the user input module 230 may be configured to output a task to modify the state of the device (e.g., an endoscope) utilized to generate procedure video data stream 202e. As another example, the user may provide an instruction to always consider the force data stream 202a. Accordingly, the user input module 230 may be configured to instruct the robotic action module 250 to always embed the force data stream 202a when generating inputs to the RT model 315. As yet another example, the user may provide inputs that the user input module 230 interpret as describing how to format the action tokens and/or detokenize the same. For example, the user may provide user input describing a relative degree by which a functionality supported by an instrument is to be activated (e.g., that a gripper should respond more strongly to operator controls, how hot a heating element should be set, etc.) to adjust the specific parameter values output by the robotic controller 325 in response to a given action token generated by the RT model 315.

In some embodiments, the modification generation model 235 may be a neural network, a LLM, or a LMM configured to accept the user inputs as an input and output one or more modifications to the system 200 that reflect the user inputs. For example, the modification generation model 235 (or other model of the system 200) may be configured to generate a constitution to provide a constitution to one or more modules 210, 220, 250, 260, and/or 270. In examples, the modification generation model 235 may analyze the data streams 202 (such as the even data stream 202b) to determine a configuration of the manipulator system (e.g., the types of components coupled to the manipulator system). In response, the modification generation model 235 may obtain a set of rules stored in a rules database (not depicted) associated with the identified component for inclusion in the various constitutions. Similarly, the modification generation model 235 may detect a user input indicative of a procedure being performed (or otherwise derive the procedure type through an analysis of the data streams 202). In response, the modification generation model 235 may obtain a set of rules stored in the rules database associated with the procedure. In some embodiments, the procedure rules may include a set of rules that generally govern the performance of the procedure and a set of rules that govern a specific phase of the procedure. In these embodiments, the modification generation model 235 may obtain the sets of rules associated with the initial phase of the procedure. After generating the constitutions, the user input module 230 may then provide the generated constitutions to the respective modules 210, 22, 250, 260, and/or 270.

The rules database may store pluralities of sets of rules including different sets of embodiment rules, procedure rules, safety rules, etc. and the modification generation model 235 may determine one or more specific sets of rules to provide to a constitution based on a user input. For example, the user input module 230 may store operator preference rules associated with specific operators or personnel, operational mode rules associated with different modes of operation (e.g., prioritizing autonomous tasks, prioritizing operator guidance or semi-autonomous tasks, prioritizing the speed of a procedure, performing a specified or specific action, etc.). In some embodiments, the modification generation model 235 may include a neural network trained to map the user inputs to specific sets of rules that implement the user input. Additionally or alternatively, the modification generation model 235 may include a generative AI model to automatically generate new rules and/or supplement a stored set of rules with additional rules based on the user inputs. It should be appreciated that the prompt to the generative AI model that generates new rules may include pre-defined instructions significantly restricting the creativity of the generative AI model to improve the likelihood the new rules are effective at restricting the disclosed models in the intended manner.

Additionally, in embodiments where the user input module 230 is coupled to a chatbot interface, the user input module 230 may determine certain responses to provide to the user to solicit user input and preferences for a given environment, situation, or procedure. For example, the user input module 230 may receive video data from the data streams 202 and may determine that an anomaly has occurred during an operation, or that required personnel is not present in an operating room for performing a procedure. The user input module 230 may then provide a response to the user via a display, or via audio to solicit a preference for performing one or more new tasks responding to the anomaly. For example, the user may indicate that a certain task should now be performed automatically via one or more repositionable structures, and in such a case, the user input module 230 may determine, based on the user input, that a new set of rules must be added to one or more constitutions (such as the task selection constitution 221) to restrict the selected tasks to be only autonomous tasks.

In other examples, the user input module 230 may determine and provide one or more responses to solicit user input based on the data streams 202, input from personnel, patient data, information pertaining to a procedure or a phase of a procedure, or other data and information. The user may then provide the solicited input in response to the provided prompts, and the user input module 230 may then identify the user input, identify any modifications, and then generate and perform the modifications to the data streams 202, and/or constitutions 211, 221, 251, 261, and 271. It should be appreciated that while constitutions 211, 221, 251, 261, and 271 are indicated by separate reference numbers, in some embodiments, a single constitution may be utilized as an input to multiple of the modules 210, 220, 250, 260, and 270.

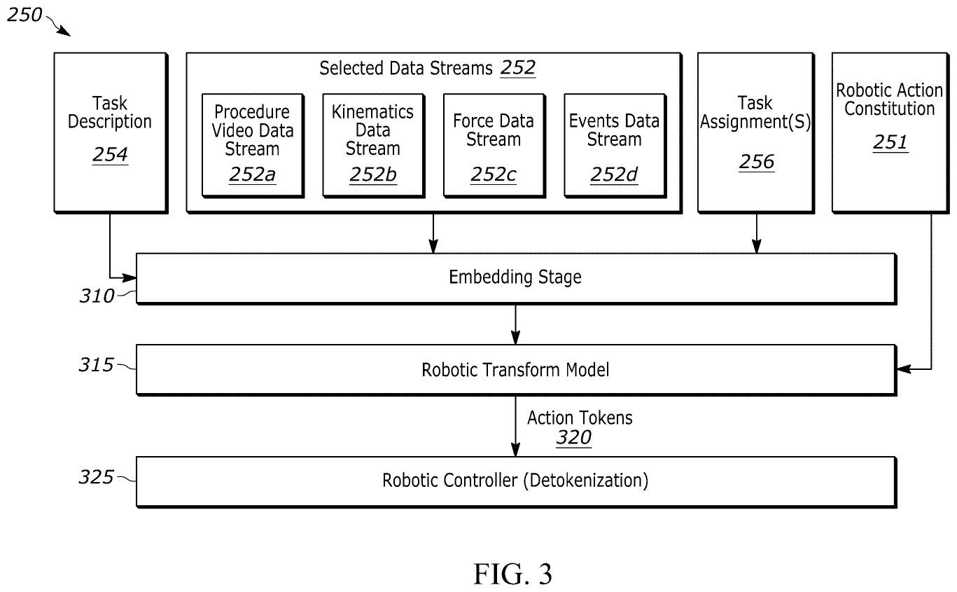

FIG. 3 is a schematic diagram of a robotic action module 250 (such as the robotic action module 250 of FIG. 2), for generating actions tokens to control a repositionable structure (such as the repositionable structures 120 of FIG. 1). The robotic action module 250 may be implemented as part of the AI assist module 180 of FIG. 1. As described with respect to FIG. 2, the robotic action module 250 may be configured to receive as inputs one or more data streams forming the multi-modal data 202 or a subset of selected data streams 252, an indication of a selected task 254, an indication of one or more specific assigned tasks 256, and a constitution such as the robotic action constitution 251 of FIG. 2.

As illustrated, the robotic action module 250 includes an embedding stage 310 that embeds the various data modalities (e.g., text, image, audio, video, sensor data, etc.) into a common data space for input into an RT model 315. The embedding stage 310 may be configured to project the input data streams into a common vector space. In examples, the embedding stage 310 may implement one or more embedding methods including, without limitation, a word2vec, GloVe, ELMo, BERT, principal component analysis, singular value decomposition, a transformer model, Doc2Vec, paragraph vectors, convolutional neural networks, pre-trained models for image embedding, node embeddings, iknowledge graph embeddings (e.g., TransE, TrensR, DistMult, etc.), word embeddings, graph embeddings, entity embeddings, or another type of embedding supported by the RT model 315. It should be appreciated that while the embedding stage 310 is depicted as a single block, each data stream 252 may be associated with a respective embedding model for generating embeddings of the respective data type included within the embedding stage 310. Further, while embedding stage 310 is depicted as a component of the robotic action module 250, in some embodiments, the embeddings generated by the embedding stage 310 are input into the various models of the task generation module 210, the task selection module 220, and the task assignment module 230. The robotic action constitution 251 may include rules for how the embedding stage 310 embeds the data streams, and which data streams 252 the embedding stage 310 embeds.

The various embedding models implemented by the embedding stage 310 may be trained and/or fine-tuned using historical data of prior procedures. As one example, an embedding model to analyze a procedure video data stream 252a may be trained to identify objects (e.g., instruments, ports, anatomical features, etc.) that are expected to be seen during the procedure. In this example, the embedding model for the procedure video data stream 252a may be trained and/or fine-tuned using historical image data in which the pixels representative of the corresponding objects are labels. The embedding model may then be trained or tuned in any suitable manner that minimizes loss with respect to the set of ground truth labels. For example, the robotic action constitution 251 may provide the rules for how the RT model 315 is to analyze the embeddings to produce a corresponding action token 320.

The robotic action module 250 then provides the embedded data output from the embedding stage 310 as an input to the RT model 315. The RT model 315 may implement any type of robotic transformer architecture configured to convert embeddings into action tokens for control of a robotic system. One such suitable architecture is the RT-2 architecture by DeepMind, but other RT model architectures are envisioned. The RT model 315 is configured to process the embedded data from the embedding stage 310 to generate action tokens 320 for controlling the instruments, auxiliary devices, and/or repositionable structures to perform the selected tasks. The action tokens 320 may be textual descriptions of robotic actions that the selected arm, instrument, and/or auxiliary device are to perform to implement the selected task. It should be appreciated that the particular tokens supported by the manipulator system may vary between models. Accordingly, the system 200 may be coupled to a library of RT models 315 each fine-tuned to generate action tokens supported by a different manipulator system type. As a result, the particular outputs of the tuned RT model 315 are adapted to the actual manipulator system associated with the system 200. The robotic action constitution 251 may define the rules for the RT model 315 for how to generate the action tokens 320 for performing a procedure. For example, the robotic action constitution 251 may include procedure rules, and embodiment rules, among other sets of rules that determine how the RT model 315 generates the action tokens 320 to perform certain tasks and procedures, given a specific manipulator system type.

The robotic action module 250 then inputs the action tokens 320 to a robotic controller 325 that de-tokenizes the action tokens 320 into actual control commands the control operation of instruments, arms, and/or auxiliary devices according to the assigned tasks 256. In examples, to perform an assigned task 256, a de-tokenized action token may include a plurality of commands including movements of one or more repositionable structures, control of an action of an instrument (e.g., power up, power down, inject, extract, incise, etc.), control of an auxiliary structure (e.g., surgical bed, display device, etc.), or another command for performing a given task. In some embodiments, the de-tokenized control commands are signals suitable for various types of robotic control architectures that may be implemented at the manipulator system. For example, the de-tokenized control commands may be a signal input into a proportional-integral-derivative (PID) controller that controls a particular joint, or a model predictive control (MPC) signal, and/or other types of control signals. As described further herein, the de-tokenized commands may be specific to a given robotic system or machine. For example, a given system may include manipulators with more degrees of freedom of motion than another system, or an instrument, such as a camera, may have a wider field of view than another camera, and the de-tokenized commands may differ depending on the various capabilities and parameters for a given robotic system.

As illustrated, in some embodiments the robotic controller 325 may also accept the robotic action constitution 251 as an input to provide rules for how actions tokens are to be converted to specific parameter values. For example, the embodiment rules may include parameter range values for controlling function degrees of freedom associated with the controlled instruments. Accordingly, by dynamically adapting the embodiment rules, the system 200 is able to ensure that the action tokens can implemented across a wide variety of instrument types without needing to fine-tine the RT model 315 for each new instrument type.

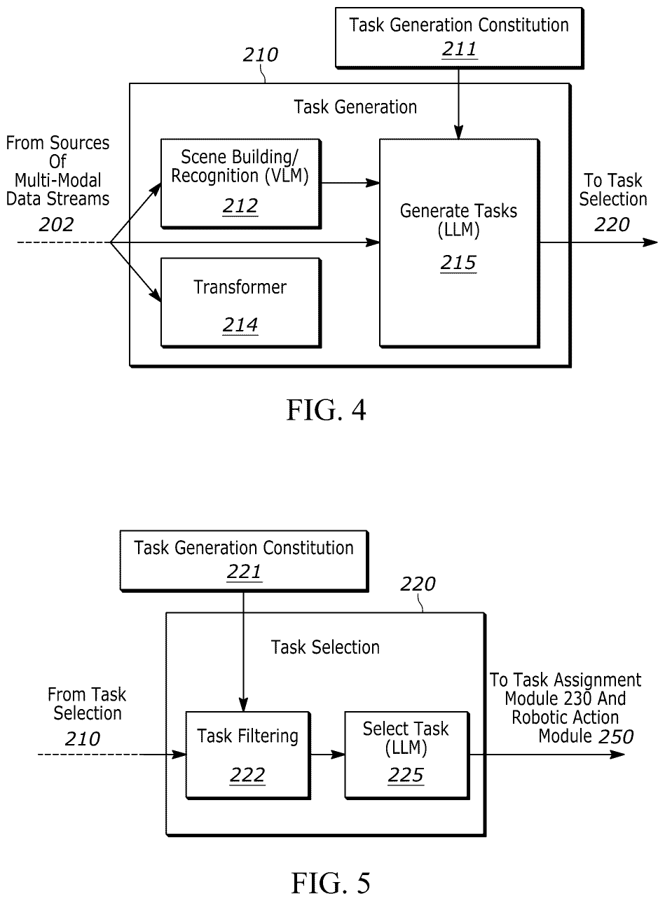

FIG. 4 is a schematic diagram of the task generation module 210 for determining tasks that can be performed based on a current state of the manipulator system and/or procedure as reflected by the multimodal data streams 202. As shown in FIG. 2, the task generation module 210 is configured to receive the multi-modal data streams 202 and the task generation constitution 211 as inputs. Similar to the embedding stage 310 of the robotic action module 250, the task generation module 210 may embed the data streams 202 for input into a task generation model 215. In some embodiments, the embedding models used to embed the data streams 202 are the same as the embedding models included in the embedding stage 310 of the robotic action module 250. The task generation constitution 251 may provide rules for how the task generation module 210 embeds the data streams 202.

For example, the embedding model for an endoscopic data stream may be routed to a vision-language model (VLM) such as scene recognition model 212 configured to output natural language descriptions of a scene depicted by the image data output by an endoscopic instrument. In surgical applications, for example, the VLM may be configured to identify objects, such as surgical instruments and devices (e.g., surgical beds or tables, medical devices, display devices, etc.), individuals and personnel (e.g., clinicians, doctors, medical technicians, etc.) depicted by an image data stream.

As another example, the embedding model of the event data stream may be configured to analyze the time series event data using a transformer model 214 to generate an output token identifying a particular phase of the procedure. It should be appreciated that while the term “transformer model” is used herein, in other embodiments, other model architectures suitable for analyzing temporal dependencies may be implemented. These output tokens may be input into the task generation model 215 to generate tasks that are in furtherance of operation typically associated with the identified phase. Similarly, the transformer model 214 may be configured to detect the completion of a phase or sub-phase of the procedure and generate an output token identifying the transition. These output tokens may be input into the task generation model 215 to begin generating tasks to implement actions associated with a subsequent phase or subphase. In some embodiments, output tokens indicative of the procedure phase may also be provided to the user input module 230 to update the constitutions with the set of procedural rules associated with the new procedure phase. Additionally, the transformer model 214 may be configured to detect anomalous operation associated with phase of the procedure and output token indicative of the anomaly (e.g., a token indicative that anomalous operation is occurring, a token indicating a particular type of anomaly that is occurring, and so on). These output tokens may be input into the task generation model 215 to generate tasks to correct the anomaly.

In addition to generating tokens based on time-dependencies in the event data stream, the transformer model 214 may also be configured to output tokens based on an instantaneous representation in the event data stream. To this end, the event data in the event data stream may indicate the complete state of the robotic components of the manipulator system at any given time. Accordingly, the transformer model 214 may be configured to identify equipment operatively coupled to the control system (e.g., manipulator arms, instruments, auxiliary devices) to generate an inventory of devices. In some embodiments, the inventory may also be input to the task generation model 215 such that the task generation model 215 understands that capabilities of the control system and generates tasks that can be implemented thereby.

In addition to identifying equipment currently coupled to the control system, an embedding model for another data stream may be configured to detect equipment that is otherwise available for use in the procedure (e.g., sterilized equipment on-hand in an operating room, equipment available elsewhere at the site). For example, the on-hand equipment may be detected via an operating room video data stream or via an equipment scheduling data stream. Additionally, the task generation module 215 may receive data indicative of personnel that are currently available, will be available for a scheduled procedure, or are required to perform one or more tasks via the data stream indicative of personnel and data associated with personnel 202f. In some embodiments, if the task generation module 210 detects the presence of new equipment, the task generation module 210 may generate an input to the user input module 230 such that the user input module 230 can update the constitutions with the appropriate embodiment rules.

The tokens generated by the embedding models may then be input into a task selection model 215. The task selection model 215 may be a LMM configured to directly accept image data streams or an LLM that accepts the natural language description(s) of the image data stream(s) generated by the VLM (e.g., scene recognition model 212). The task selection model may then generate tasks in furtherance of the detected (or subsequent) phase of the procedure based on the available equipment, personnel, and any other conditions indicated by the input data streams 202. The task generation constitution 211 may include sets of rules that control how the task selection model 215 generates the tasks from the detected phase of procedure, available equipment, personnel, and other conditions detected or indicated by the input data streams 202.

It should be appreciated that the task generation model 215 may be configured to output multiple tasks to implement the actions associated with a particular phase of the procedure. For example, to implement a procedural phase associated with advancing the instruments to a worksite, the task generation model 215 may generate tasks associated with aligning the instruments with respective ports and advancing the instruments towards the worksite. It should be appreciated that the task generation model may be configured to generate multiple different sets of tasks for implementing the procedural phase. To this end, the different sets of tasks may be performed to implement a procedural phase. Similarly, the tasks within a set of tasks may be performed in different sequences. This enables the various different approaches to performing a procedural phase to be evaluated such that an optimal set of tasks is ultimately selected for implementation.

FIG. 5 is a schematic diagram of the task selection module 220 configured to select one or more tasks and/or sets of tasks generated by the task generation module 210 for implementation by the control system. As illustrated, the task selection module 220 may select the tasks in two stages. As shown in FIG. 2, the task selection module 220 is configured to receive the generated tasks from the task generation module 210 and the task selection constitution 221 as an input.

In the first stage, a task filtering model 222 is implemented to categorize the generated tasks for filtering. For example, the task filtering model 222 may categorize each task is capable of being performed autonomously, semi-autonomously, manually, or not at all. In some embodiments, the constitution 221 includes rules that define how to classify different categories of tasks as being capable of autonomous, semi-autonomous, or manual performance. Tasks that are not capable of being performed may be filtered out. It should be appreciated that while the task generation model 215 is generally configured to generate tasks in view of the configuration of the manipulator system, the generative nature of LLMs and LMMs may still result in the generation of tasks that cannot be performed. Accordingly, the task filtering model 222 may function as a sanity check mechanism on the task generation model 215 to ensure only feasible tasks are implemented.

In a second stage, the remaining tasks are input into a task selection model 225 via a prompt that also includes the task selection constitution 221. In some embodiments, the task selection model 225 is an LLM. The task selection model 225 is configured to analyze the potential tasks or sets of tasks to be performed and select a preferred task or set of tasks to implement. For example, the task selection model 225 may be configured to evaluate the tasks or set of tasks using one or more evaluation metrics (e.g., time to perform the task, confidence in ability to autonomously perform the task, an ease of manual implementation, a confidence in safe performance of the task, abilities or personnel, available personnel, or relevant tasks).

In examples, the task selection constitution 221 may include user preference rules about the types of tasks (or the sequence of tasks) that a user prefers to perform. Accordingly, by inputting a prompt that includes the task selection constitution 221 into the task selection model 225, the tasks selected by the task selection model 225 will generally comply with the user preferences. In other examples, the system 200 may detect that a component is malfunctioning, is in a low-life state, and/or should otherwise not be utilized when performing the procedure. In these situations, the user input module 230 may dynamically alter the embodiment rules and/or the safety rules included in the task selection constitution 221 to prevent the tasks that require the component from being selected.

The task selection model 220 then outputs the selected task(s) and provides the selected task(s) to the task assignment module 230, and the robotic action module 250.

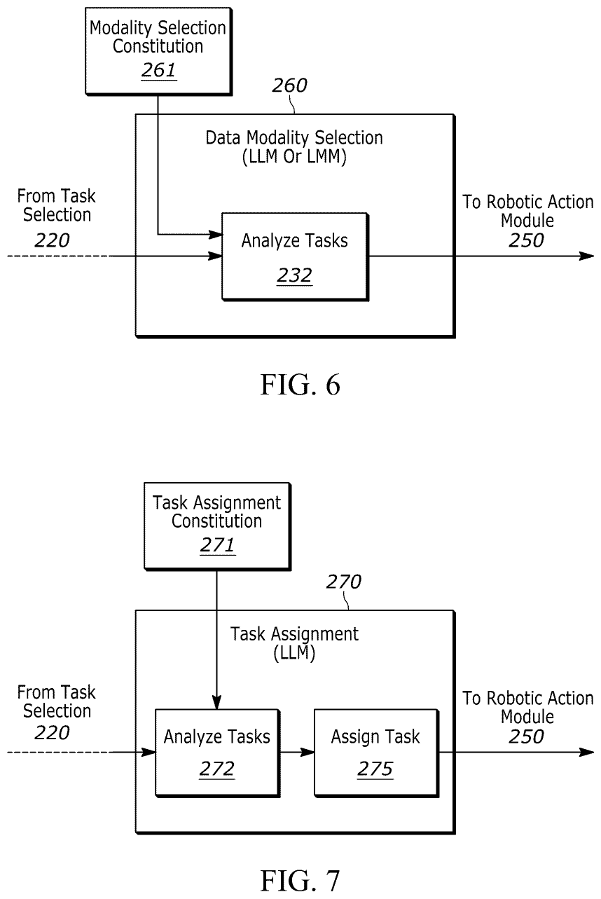

FIG. 6 is a schematic diagram of an example data modality selection module 260 of FIG. 2 for determining task specific data streams for controlling a repositionable structure (such as the repositionable structures 120 of FIG. 1) to perform a task (such as the tasks selected by the task selection module 220). Accordingly, the data modality selection module 260 may be communicative coupled to the task selection module 220. The data modality selection module 260, as shown in FIG. 2, receives the tasks from the task selection module 220 and the modality selection constitution 261 as inputs.

As illustrated, the data modality selection module 260 may include a task analysis model 262 configured to identify the data modalities required to perform an input task. The task specific data modalities may include one or more streams of data including, without limitation, one or more of image, video, audio, force feedback, user input, event, or instrument provided data streams. In some embodiments, the task analysis model is an LLM. In these embodiments, the task analysis model 262 may be configured to generate a prompt to the LLM that asks the LLM to identify the data streams required to perform the input task. In some embodiments, the input includes a description of the available multi-modal data streams 202 and/or a state thereof, as well as the modality selection constitution 261. Additionally, the input may include a description of additional data streams that are not currently available and/or alternative states of the available data streams that may be achieved.

The modality selection constitution 261 may include sets of rules that govern how the task analysis model 262 identifies the various data modalities to perform an input task and for generating the prompt to the LLM. For example, the modality selection constitution 261 may include a set of procedural rules that define specific tasks and required data types for each given task and the task analysis model may identify the data modalities based on the required data types and tasks as defined by the procedural rules. Additionally, the modality selection constitution 261 may include phase specific rules that instruct the task analysis model on how to analyze the various data modalities to determine phase-specific data modalities required for various phases of a procedure.

Based on the inputs, the task analysis model 262 may be configured to output a set of data modalities required to perform implement the task and associate the input task with the corresponding set of data modalities. For example, the task analysis model 262 may identify a type of task associated with the input task (e.g., a repositioning task, an end effector activation task, a configuration task, etc.) to identify which data modalities are required to perform the input task such that non-necessary data modalities can be ignored when implementing the task thereby providing the above-described improvements in execution time by the robotic action module 250. For example, endoscopic image data may not be required to perform a task related to mounting a new instrument to a robotic arm. As another example, operating room image data may not be required to ablate a target anatomy. As yet another example, a task to change the state of an auxiliary device may not require kinematic data associated with manipulator system.

If the required data modalities are available, the data modality selection module 260 may then output the task and data modalities to the robotic action module 250 for implementation at the appropriate time. If the required data modalities are not available, the modality selection constitution 261 may include rules that cause the data modality selection module 260 to generate an output to the task generation module 210 instructing the task generation module 210 of the need to make the additional data modalities available. In some embodiments, the output may be a plain text instruction (e.g., “enable endoscope video feed,” “couple endoscope to instrument X,” or “move endoscope to include view of target anatomy”). In response, the task generation module 210 may generate the instructed tasks to autonomously (or semi-autonomously) make the indicated data modalities available. It should be appreciated that the data modality selection module 260 or the robotic action module 250 may queue the originally input task until detecting that the task to make the required data modality available has been successfully performed.

FIG. 7 is a schematic diagram of an example task assignment module 270 of FIG. 2 for assigning manual and/or semiautonomous tasks (such as the tasks selected by the task selection module 220) associated with a medical procedure. Accordingly, the task assignment module 270 may be communicative coupled to the task selection module 220. As shown in FIG. 2, the task selection module 220 is configured to receive the generated tasks from the task generation module 220 and the task assignment constitution 271 as an input.

As illustrated, the task assignment module 270 may include a task analysis model 272 configured to analyze the selected tasks and determine personnel to perform the selected tasks according to sets of rules of the task assignment constitution 271. In some embodiments, the task analysis model 272 is an LLM. In these embodiments, the task analysis model 272 may be configured to generate a prompt to the LLM that asks the LLM to identify the various personnel, equipment, robotic systems, etc. required to perform the input task, as well as the task assignment constitution 271. For example, the task analysis model 272 may identify a type of task associated with the input task (e.g., a repositioning task, an end effector activation task, a configuration task, etc.) and identify which equipment and/or personnel are required to perform the input task. As one example, an anesthetist may be required for performing certain tasks (administering the anesthesia), while the anesthetist is not required for other tasks (manually repositioning a manipulator).

After identifying the requirements to perform the task and/or subtasks thereof, the task assignment module 270 may input a prompt that includes the tasks, the data streams 202, and the task assignment constitution 271 into the task assignment model 275. It should be appreciated that the task assignment model 275 may be the same or different LLM as the task analysis model 272. In embodiments where the same LLM is used for both models 272, 275, the functionality of the models 272, 275 may be achieved via a single prompt to the LLM. In these embodiments, the task assignment constitution 271 input to the LLM with the prompt may include additional rules that govern the task assignment process.

In examples, the task assignment constitution 271 includes a set of rules defining how the LLM is to analyze the tasks to identify the required equipment and/or personnel for each task. As one example, the task assignment constitution 271 may include rules that define how to select between multiple instruments capable of performing a task. As one example, the task assignment constitution 271 may include rules the state that the instrument that has to move the least to accomplish the task should be assigned the task. As another example, the task assignment constitution 271 may include user preference rules that define how the user prefers the tasks be assigned (e.g., to a manipulator arm and/or instrument teleoperated by the user's dominant hand). Accordingly, when the input task and the constitution 271 are input to the LLM, the LLM may output the task assignment that complies with the user preferences.

In some embodiments, if the task involves semi-autonomous implementation (or an autonomous implementation with an assigned instrument), the task assignment module 270 may provide an indication of the task to the robotic action module 250 for implementation at the appropriate time.

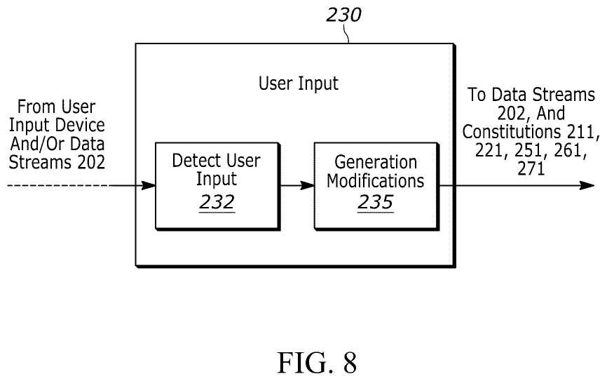

FIG. 8 is a schematic diagram of an example user input module 230 of FIG. 2 for detecting user input and modifying one or more data streams 202 and/or constitutions 211, 221, 251, 261, and 271 according to the user input. As shown in FIG. 2, the user input module 230 is configured to receive one or more user inputs and preferences from a user interface or device, or may receive a data stream 202g indicative of a user input or preference or of additional patient data (e.g., operation or health history, current patient diagnosis and/or conditions, etc.).

As illustrated, the user input module 230 includes a detect user input model 232 that analyzes the user input data stream and data from one or more user input devices, and determines that a user input has been provided. The detect user input model 232 analyzes the user input to determine what types of user input are provided, and what, if any, modifications to the data streams 202 and/or constitutions 211, 221, 251, 261, and 271 should be performed.

The user input module 230 further includes a modification generation model 235 that receives the determined types of user input and determined modifications from the detect user input model 232. The modification generation model 235 then generates the determined modifications and provides, or performs, the modifications to the data streams 202, and/or constitutions 211, 221, 251, 261, and 271. The modification generation model 235 may modify the data streams 202 by enabling/disabling the data sources associated with the data streams and/or by configuring the data sources to capture a particular set of data (e.g., by changing a field of view of an image sensor). Additionally, the modification generation model 235 may modify one or more of the constitutions 211, 221, 251, 261, and 271 by editing the constitution to add rules, remove rules, or further edit the rules and natural language text of a constitution.

As described above, the user input module 230 may further generate and initialize a constitution based on user input. For example, the user input module 230 may initialize at least one of the constitutions 211, 221, 251, 261, and/or 271, and the modification generation model 235 may generate the rules and natural language text to include in the constitutions. The user input module 230 may initialize any of the constitution and include any types of sets of rules in each constitution independently. For example, the user input module may initialize and/or edit any of the constitutions 211, 221, 251, 261, and 271, to include one or more sets of foundational rules, safety rules, embodiment rules, and/or user preference rules.

The foundational rules may include rules pertaining to the allowable robotic actions and limitations of robotic actions. For example, the foundational rules may define rules that prevent robotic actions from being performed that could harm a human or operator, rules that cause a robotic system to receive and follow orders provided by users, prevent a robotic system from harming a patient, prevent a robotic system from interfering with other devices and tasks, etc.

The safety rules may include rules that define actions and operations that are deemed safe for a given environment, operation, and scenario. For example, the safety rules may include rules that restrict or define spatial limitations to movement or operation of a robotic system, rules that pertain to maintaining sterility of a robotic system or instruments, rules that define safe use of specific instruments and devices, rules that limit a robotic systems abilities pertaining to known limits of a specific robotic system model, etc. The safety rules may be specific to a scenario or environment such as a certain institution may have different safety standards than other medical facilities and therefore the safety rules may reflect the various standards of institutions, states, regions, governments etc. Additionally, the safety rules may also be specific to certain personnel present or performing tasks for an operation. For example, the safety rules may depend on the types of personnel and training of personnel available for performing an operation.

The embodiment rules may include rules that define capabilities and limitations pertaining to specific models of instruments, devices, and systems. For example, the embodiment rules may include rules pertaining to available degrees of freedom of motion, ranges of motion, imaging resolutions, video capabilities, audio recording parameters, etc. The embodiment rules may include rules that pertain to the dimensions and sizes of various instrument, device, or system components to control how a robotic system or instrument moves in an environment, and to prevent components from contacting other objects as required. Additionally, the embodiment rules may include rules that specify and govern actions that may be performed by specific instruments, devices, and systems such as rules that control insection, suction, imaging, injection, extraction, application of radiation, heating, etc.

The user preference rules may include rules pertaining to rules that are defined by a user or based on user input and preferences. For example, the user preference rules may include rules that prioritize use one instrument or device over another based on a user provided preference, that prioritize use of a specific robotic arm or a manipulator system, control how one or more models (such as the task selection model 220) filters tasks into automatic, semi-automatic, and manual tasks based on user input, how the task assignment model 270 assigns tasks, what is displayed to a user, how data and prompts are displayed to a user, etc.

As another example, a set of rules may relate to safety and autonomy settings. For example, when performing a challenging or high-risk procedure, users may provide user inputs indicating that the system should reduce autonomy and leave more tasks for the human operator to perform. On the other hand, for easy or routine procedures, the user may increase the autonomy of the robotic system to improve workflow efficiency. Accordingly, the rules in the task assignment constitution 271 may be adapted to such that the actor assigned to the generated tasks are aligned with the autonomy preferences indicated by the user.

While described as various specific sets of rules above, other sets of rules are envisioned to be included in the constitutions 211, 221, 251, 261, and 271. Additionally, any additional types and sets of rules may be generated by the user input module 230 and edited to be included in any of the constitutions.

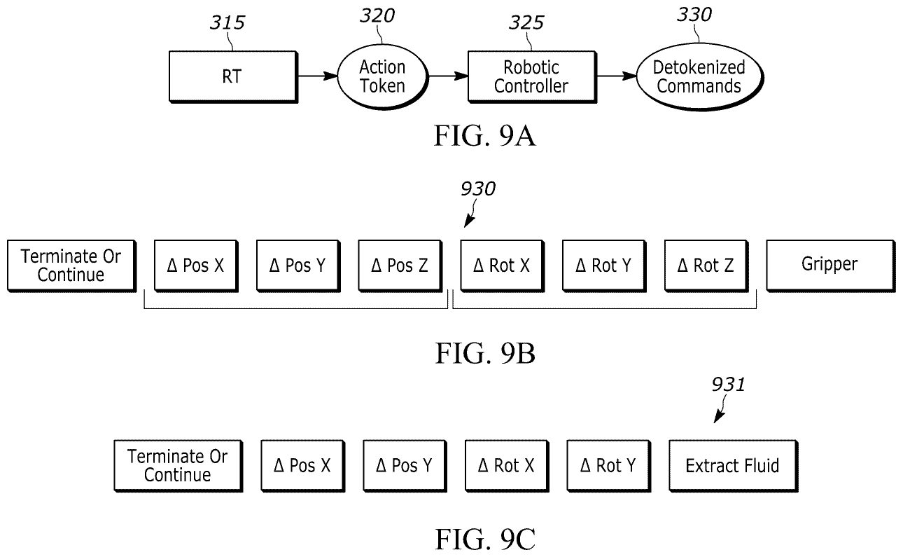

FIG. 9A is a schematic diagram illustrating detokenizing action tokens to control a repositionable structure (such as the repositionable structures 120 of FIG. 1), an instrument, or an auxiliary device to implement a semi-autonomous task. Detokenization is a process under which a system converts an action token 320 (e.g., a text string or commands) output by the robotics transformer model 315 into actual robotic commands 330 (an “action sequence”) to control the indicated equipment, for example, by changing the pose or by activating or otherwise controlling a functionality supported by the indicated equipment.

As described above, the RT model 315 may be fine-tuned and/or selected based on the particular manipulator system model. In examples, the robotic action constitution 251 may be input to the RT model 315 and include various sets embodiment rules that pertain to different manipulator system models. The robotic action constitution may instruct the RT model 315 to use specific model rules according to a corresponding manipulator system in use. Additionally, the user input module 230 may identify a particular manipulator system model and edit the robotic action constitution 231 to include the corresponding rules for operating the particular manipulator system model. In examples, knowledge of the capabilities of the manipulator system are incorporated into the RT model 315 itself and/or in the robotic action constitution 251. Similarly, the RT model 315 may be configured to maintain and/or access a list of current equipment coupled to the control system. Accordingly, the action tokens 320 output by the RT model 315 may include a textual indication of the equipment that is to perform the action. For example, an output action token 320 may state “Move Instrument X coupled to Arm A to target work area” or “Use Instrument Y to cauterize incision.” Accordingly, the action tokens 320 and detokenization are specific to a robotic system or model based on the capabilities of a given robotic system.

In some embodiments, the action tokens 320 may be further tailored to the current state of the manipulator system. For example, the RT model 315 may be configured to accept a kinematic data stream as an input such that the output action tokens indicate a pose for the instrument and/or arm in the robotic coordinate system. Accordingly, as one example, rather than outputting an action token 320 indicating that an instrument should move to the target work area, the output action token 320 may instead indicate “Move Instrument X to position (x, y, z) and orientation (α, β, γ).” As a result, the action tokens 320 output by the RT model 315 are tailored to the specific state of the manipulator system improving the ability of the control system to accurately interpret and implement autonomously-generated control commands.

Similarly, because the RT model 315 is aware of the functionality supported by the instruments coupled to the control system, the RT model 315 is able to output commands that are specifically tailored to the implementing instrument. For example, rather than outputting an action token that states “Clamp blood vessel,” the RT model 315 may instead output an action token 320 that states “Place gripper around blood vessel at position (x, y, z) and engage grip to exert 50 pascals of force.” In these embodiments, the robotic action constitution 251 may include embodiment rules that define the amount of force (or other functional DOF parameter value) to implement the task. As a result, adapting the robotic action constitution 251 input into the RT model 315 enables the generation of action tokens 320 that have improved alignment with the actual functionality supported by the control system, thereby enabling more precision in the generation of the action tokens 320 and more robust usage of instrument functionality.

After the RT model 315 generates the action tokens 320, the action tokens 320 are input into the robotic controller 325 to convert the natural language action tokens into de-tokenized commands 330 (e.g., parameterized commands, such as PID parameter values or MPC parameter values, used by the control system actually realize the instructed actions). With simultaneous reference to FIGS. 9B and 9C, depicted are example parameterized command structures utilized by the control system. In some embodiments, the robotic action constitution 251 is also input into the robotic controller 325 to provide rules for how to convert an action token into a parametric control value. For example, a user may indicate that the default action tokens for a gripping command exert too little pressure and include a user preference rule to scale up the output value for the pressure DOF by 5%.

The de-tokenized command 930 of FIG. 9B is configured to control operation of a gripper instrument that has 6 motive degrees of freedom (DOFs). Accordingly, the de-tokenized command 930 may include deltas by which the control system is to change each DOF. Additionally, the de-tokenized command 930 includes additional DOFs related to the functionality supported by the instrument. For example, in the illustrated example related to a gripper, the “gripper” DOF may indicate an amount of force the gripper should exert. Similarly, the de-tokenized command 931 of FIG. 9C is configured to control operation of a fluid extraction instrument that has 5 motive degrees of freedom (DOFs) and one functional DOF. It should be appreciated that the length of the de-tokenized control commands 330 may vary based on the number of motive and functional DOFs supported by the controlled equipment. Accordingly, the length of the de-tokenized commands 330 have fewer unused parameters, thereby enabling more efficient usage of control buses.

In some embodiments, the de-tokenized commands 330 may also control one or more user feedback devices, such as a display device. For example, a de-tokenized command 330 may be configured to cause a display unit to provide an instruction for executing a task that is to be performed manually or semi-autonomously by a user.

While FIGS. 2-9C describe a process for performing task assignment to personnel, instruments, and robotic systems to implement a one or more tasks, it should be appreciated that the disclosed process may be repeated throughout the procedure until its completion. As a result, each stage of the procedure may be discretized into tasks, that are converted into de-tokenized commands such that any portion of the procedure can be implemented via closed-loop autonomous control, semi-autonomous control, or manually by personnel. It should be appreciated that in some embodiments, the modules 210, 220, and/or 230 may analyze the multimodal data stream 202 to anticipate future tasks, and assign the future tasks, that are predicted to be performed based on a current state. Thus, the tasks analyzed by the modules 210, 220, and/or 230 may differ from the task being implemented by the robotic action module 250 or by personnel. As a result, the control system is able to anticipate future actions and future task assignments in order to ensure closed-loop control of the overall procedure occurs more efficiently.

In some embodiments, the control system may further include a scheduler module configured to generate a sequence of expected tasks (and their corresponding data modalities and/or assigned personnel, instruments, robotic systems, or auxiliary devices) and their corresponding triggers for execution. Accordingly, when the scheduler detects, based on the multimodal data streams 202, that a trigger event has occurred, the scheduler can route one or more subsequent tasks to robotic action module 250 or provide indications of a specific task to respectively assigned personnel. It should be appreciated that if the multimodal data streams 202 indicate that conditions have changed (e.g., an anomalous condition is detected), the scheduler can adjust the priority and/or sequencing to prioritize addressing the current conditions.

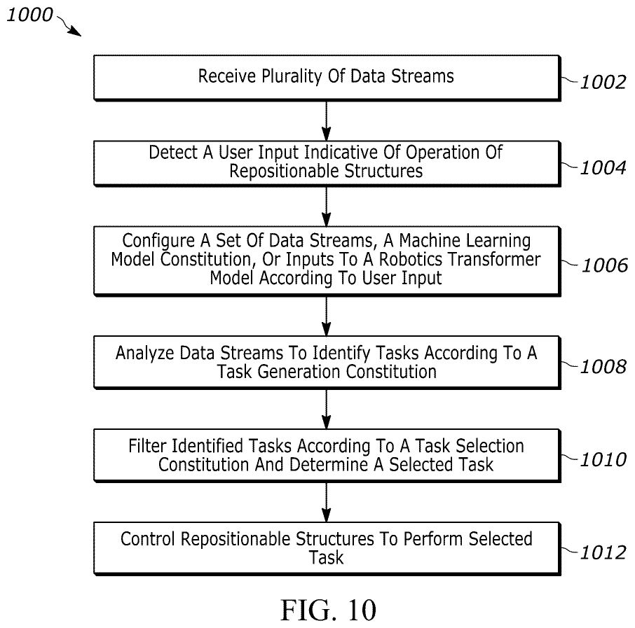

FIG. 10 is a flow diagram of a method 1000 for determining and performing tasks for a medical procedure using adaptive AI based on user input and user preferences. The method 1000 may be performed by a processor system or a control system (such as the processor system 150 and control system 140 of FIG. 1). In some embodiments, the control system may implement an AI-assist module (such as the AI-assist module 180) to perform the functionality described with respect to the machine learning models. In implementations, the method 1000 may further be performed with a system including one or more repositionable structures that may be operably coupled to one or more instruments (such as the instruments 122).

The method 1000 may begin at block 1002 when the control system receives a plurality of data streams (such as the multimodal data streams 202) from one or more data sources. The data streams may include multi-modal data streams of different types of data, as provided by different devices or systems. For example, the multi-modal data streams may include data from a video camera, audio data, force sensor data, system events (such as the events data stream 252b), endoscopic image data (such as the procedure video data stream 252e), operating room image data (such as the external video data stream 202d), kinematics data (such as the kinematics data stream 252c), haptics data, force data (such as the force data stream 252a), shape sensing data, tissue impedance data, environmental data, personnel procedure history (such as the personnel data stream 202f), phase information, task information, personnel training data, a data stream indicative of available instruments, intraoperative image data, and user input data (such as the user input data stream 202g). Accordingly, the one or more data streams may include environmental data including one or more of data indicative of an interaction between the system and its environment, force data associated with instrument contact with patient tissue, force data associated with feedback to one or more manipulators, personnel present in a medical environment, and data indicative of system collisions with objects in the environment. The one or more data streams may include data indicative of schedules of personnel to determine at what times personnel may be available to perform tasks associated with a procedure. In some embodiments, the one or more data streams may include user preference data in the user input data that is indicative of user selections or preferences for a given operation, environment, or other preference. Additionally, the user input data may be indicative of a specific patient status or condition.

At block 1004, the control system detects a user input indicative of operation of the repositionable structures. For example, the user input may include a user preference, patient specific information, or data derived from the user input. The user input may be analyzed to determine a modification to and/or configuration of one or more of (i) which data streams are included in the plurality of data streams, (ii) a task generation constitution configured to modify how a task generation machine learning model generates tasks, and (iii) an input into a robotics transformer model configured to generate outputs used to control of the one or more repositionable structures or instruments. A machine learning model, such as the models of the user input module 230 of FIG. 2, may receive the user input via a user interface, or via one or more sensors and the data streams, and the user input module may identify one or more modifications to data streams, constitutions, or inputs to the robotics transformer model based on the user input.

At block 1006, the control system then configures, based on the user input, at least one of (i) which data streams are included in the plurality of data streams, (ii) a task generation constitution configured to modify how a task generation machine learning model generates tasks, and (iii) an input into a robotics transformer model configured to generate outputs used to control of the one or more repositionable structures or instruments. Additionally, the control system may perform modifications to any of the constitutions described here (such as one of the constitutions 211, 221, 251, 261, 271). In some embodiments, the actions of block 1006 may be performed by the user input module 230 of FIG. 2.

At block 1008, the control system may analyze, via a task generation machine learning model, one or more data streams from the plurality of data streams to identify a plurality of tasks to be performed by the one or more repositionable structures. As described herein, there generated tasks may be natural language outputs relating to one or more actions to be performed in furtherance of the medical procedure. Additionally, the control system may identify one or more tasks to be performed by an instrument operatively coupled to one or more repositionable structures or by a user in a manual or semi-autonomous manner.

To analyze the plurality of data streams, the control system obtains or generates a task generation constitution (such as the task generation constitution 211 of FIG. 2) for input into the task generation machine learning model (such as the task generation model 215 of FIG. 2). The task generation constitution may include one or more sets of rules that govern how the task generation machine learning model analyses the data streams and generates the natural language output relating to the one or more tasks to be performed. For example, the task generation machine learning model may include foundational rules that define allowable robotic actions (e.g., limitations on instrument operation for patient and user safety, such as Asimov's three rules), safety rules that define safe operation of the computer-assisted medical system (e.g., spatial limitations, sanitization standards, etc.), embodiment rules that define capabilities and limitations of the computer-assisted medical system, associated instruments and devices (e.g., degrees of freedom of robotic arm movement, imaging capabilities of endoscopes and imaging devices, volume capabilities of extraction and injection devices, etc.), and/or user preference rules (e.g., rules defined and input by a user or personnel for performing a procedure or task). Accordingly, the task generation machine learning model may generate the natural language outputs according to the set of rules of the task generation constitution.

As described herein, inputting the plurality of data streams into the task generation machine learning model may include generating an embedding of a first data stream (such as via the embedding stage 310) describing the medical procedure or a phase of the medical procedure. For example, the control system may provide an event data stream into a transformer model (such as the transformer model 214) trained to identify event or procedural milestones. Accordingly, an embedding of the first data stream may be a natural language description of the medical procedure and/or phase thereof. Additionally, in some examples, the control system may analyze the data streams to determine one or more current tasks being performed and, based on the current task being performed, generate one or more additional tasks to be performed.

At block 1010, the control system may filter, via a task selection machine learning model (such as the task selection model 225 of FIG. 2), the identified tasks to select specific tasks based on the output of the task selection machine learning model. The task selection machine learning model may further filter the tasks may be selected based on the tasks generated from the task generation machine learning model and any additional inputs. To filter and select the tasks, the control system may input, into the task selection machine learning model, a task selection constitution (such as the task selection constitution 221 of FIG. 2) defining a set of rules for filtering the tasks, and ultimately selecting a set of tasks to be performed. For example, the task selection constitution may include qualifications rules that indicate skills required to perform a task and/or personnel rules that indicate skills associated with personnel in the medical environment, rules for instruments required to perform a given task, rules pertaining to user preferences for performing specific tasks automatically, semi-automatically, manually, or not at all, or another set of rules for selecting the tasks. In some embodiments, the control system adaptively updates, such as by the user input module 230 of FIG. 2, the rules of the task selection constitution actively as personnel enter and exit the medical environment, as instruments become available or are determined sterile or not, or as tasks are completed.

At block 1012, the control system may be configured to control the repositionable structures to perform the selected task. To control the repositionable structures, the control system may generate, based on the set of task-specific data streams and a constitution (such as the robotic action constitution 251 of FIG. 2) and via a machine learning model (such as the RT 315 of FIG. 2), one or more action tokens (such as the action tokens 320) for controlling the one or more repositionable structures. In some scenarios, an action token may be configured to cause a display unit to present instructions to a user for performing a manual or semi-autonomous task. To control the one or more repositionable structures, the control system may further de-tokenize the action tokens into control commands (such as the de-tokenized commands 330, 830, 831). As previously described, the specific de-tokenized control commands may be specific for a given system, instrument, repositionable structure, or auxiliary device according to sets of rules, such as embodiment rules, in the robotic action constitution. De-tokenizing the action tokens into control commands may result in control commands with varied lengths depending on the given functionalities and capabilities supported by each individual device and equipment in an environment (e.g., an operating room). For example, the de-tokenized commands may vary in length depending on the number of degrees of freedom (DOFs) and functionalities supported by repositionable structures and/or instruments.

As described herein, the method 1000 may be performed using various systems including different robotic system models with different capabilities. The ability of the control system to modify the data streams, machine learning models (via constitutions), and control of instruments, devices, and repositionable structures of robotics systems allows for the implementation of robotic systems in performing medical procedures across a broad range of environments and specific instances and conditions. Inclusion of the user input module for analyzing user input and modifying the system 200 enables adaptive AI for use in generating, filtering, and performing tasks via robotic systems in medical settings may overcome the need to retrain systems to specific environments and scenarios which reduces onboarding time of new devices and instruments as well as increases the efficiency and availability of operations and treatments.

One or more components of the examples discussed in this disclosure, such as control system 140, may be implemented in software for execution on one or more processors of a computer system. The software may include code that when executed by the one or more processors, configures the one or more processors to perform various functionalities as discussed herein. The code may be stored in a non-transitory computer readable storage medium (e.g., a memory, magnetic storage, optical storage, solid-state storage, etc.). The computer readable storage medium may be part of a computer readable storage device, such as an electronic circuit, a semiconductor device, a semiconductor memory device, a read only memory (ROM), a flash memory, an erasable programmable read only memory (EPROM); a floppy diskette, a CD-ROM, an optical disk, a hard disk, or other storage device. The code may be downloaded via computer networks such as the Internet, Intranet, etc. for storage on the computer readable storage medium. The code may be executed by any of a wide variety of centralized or distributed data processing architectures. The programmed instructions of the code may be implemented as a number of separate programs or subroutines, or they may be integrated into a number of other aspects of the systems described herein. The components of the computing systems discussed herein may be connected using wired and/or wireless connections. In some examples, the wireless connections may use wireless communication protocols such as Bluetooth, near-field communication (NFC), Infrared Data Association (IrDA), home radio frequency (HomeRF), IEEE 502.11, Digital Enhanced Cordless Telecommunications (DECT), and wireless medical telemetry service (WMTS).

Various general-purpose computer systems may be used to perform one or more processes, methods, or functionalities described herein. Additionally or alternatively, various specialized computer systems may be used to perform one or more processes, methods, or functionalities described herein. In addition, a variety of programming languages may be used to implement one or more of the processes, methods, or functionalities described herein.

While certain examples and examples have been described above and shown in the accompanying drawings, it is to be understood that such examples and examples are merely illustrative and are not limited to the specific constructions and arrangements shown and described, since various other alternatives, modifications, and equivalents will be appreciated by those with ordinary skill in the art.

What is claimed is:

1. A computer-assisted system for performing automated tasks, the system comprising:
one or more repositionable structures configured to support respective instruments; and
a control system operably coupled to the repositionable structure, wherein the control system is configured to:
receive a plurality of data streams from one or more data sources;
analyze, via a task generation machine learning model, the data streams to identify a plurality of tasks to be performed by the one or more repositionable structures, wherein a task generation constitution is input into the task generation machine learning model to control how the task generation machine learning model analyzes the data streams;
filter, via a task selection machine learning model, the identified tasks, wherein a task selection constitution is input into the task selection machine learning model to control how the task selection machine learning model filters the identified tasks;
detect a user input indicative of at least (i) one of the plurality of data streams, (ii) one or more machine learning models, or (iii) the operation of the repositionable structures;
based on the user input, configure or modify at least one of:
which data streams are included in the plurality of data streams;
the task generation constitution to modify how the task generation machine learning model generates tasks, and
an input into a robotics transformer model configured to generate outputs used to control the one or more repositionable structures or instruments;
select a task to be performed based on an output of the task selection machine learning model; and
control, based on one or more outputs of the robotics transformer model, the repositionable structures to perform the selected task.

2. The computer-assisted system of claim 1, wherein, based on the user input, the control system is further configured to:
configure the task selection constitution to modify how the task selection machine learning model filters the identified tasks.

3. The computer-assisted system of claim 1, wherein:
the control system is further configured to determine, via a modality selection machine learning model and based on the task to be performed, a set of task-specific data streams; and
based on the user input, the control system is further configured to configure a modality selection constitution that is input into the modality selection machine learning model to modify how the modality selection machine learning model determines the set of task-specific data streams.

4. The computer-assisted system of claim 1, further comprising, a user input processing module configured to:
receive the user input and input the user input into a user input machine learning model trained to generate the one or more configurations or modifications of one or more of (i) a data stream of the plurality of data streams, (ii) the task generation machine learning model, and (iii) the robotics transformer model,
wherein the user input machine learning model is configured to convert the one or more configurations or modifications into a control signal that implements the one or more configurations or modifications.

5. The computer-assisted system of claim 1, further comprising, a user input processing module configured to:
receive the user input and input the user input into a user input machine learning model trained to generate the one or more configurations or modifications of one or more of (i) a data stream of the plurality of data streams, (ii) the task generation machine learning model, and (iii) the robotics transformer model,
wherein the control system is further configured to:
store sets of rules associated with different types of operations of the repositionable structures; and
the user input machine learning model is configured to:
identify an indicated set of rules based on the user input.

6. The computer-assisted system of claim 5, wherein a set of rules of the stored sets of rules corresponds to operator preference rules and the user input is indicative of an operator identifier.

7. The computer-assisted system of claim 5, further comprising:
configuring a constitution by adding the indicated set of rules to the constitution or replacing a set of rules with the indicated set of rules.

8. The computer-assisted system of claim 5, wherein the types of operation include one or more of a mode to prioritize autonomous tasks, a mode to prioritize operator guidance, a mode to prioritize procedure speed, and a mode to perform an indicated action.

9. The computer-assisted system of claim 1, further comprising, a user input processing module configured to:
receive the user input and input the user input into a user input machine learning model trained to generate the one or more configurations or modifications of one or more of (i) a data stream of the plurality of data streams, (ii) the task generation machine learning model, and (iii) the robotics transformer model,
wherein the control system is further configured to assign, according to a task assignment module, actions to components of the one or more repositionable structures to implement the selected task.

10. The computer-assisted system of claim 9, wherein the user input machine learning model is further configured to:
identify one or more modifications to the task assignment module, wherein the modification to the task assignment module includes a change in user preference data input to the task assignment module.

11. The computer-assisted system of claim 1, wherein the plurality of data streams includes one or more of endoscopic image data, operating room image data, kinematics data, haptics data, force data, shape sensing data, environmental data, intraoperative imaging, personnel identification, personnel procedure history, personnel training.

12. The computer-assisted system of claim 1, wherein the task generation constitution and the task selection constitution include foundational rules, safety rules, embodiment rules and procedural rules.

13. The computer-assisted system of claim 1, wherein the user input includes natural language text.

14. The computer-assisted system of claim 13, further comprising:
a microphone configured to record audio data;
wherein the natural language text is derived from the audio data.

15. The computer-assisted system of claim 1, further comprising:
a display device;
wherein the user input is generated based on a user interaction with a user interface presented on the display device, and
wherein the user interaction includes an indication associated with intraoperative or preoperative image data presented by the display device.

16. The computer-assisted system of claim 1, wherein the control system is further configured to:
analyze event data identifying the repositionable structures or components of the repositionable structure; and
configure at least one of the task generation constitution or the task selection constitution based on the identified repositionable structures or components.

17. The computer-assisted system of claim 16, wherein to configure the constitution, the control system is configured to:
identify a set of embodiment rules corresponding to the identified repositionable structures or components; and
add the identified set of rules to the task generation constitution or the task selection constitution.

18. The computer-assisted system of claim 1, wherein:
the control system is further configured to:
analyze the plurality of data streams to identify a procedure being performed; and
configure the task generation constitution or the task selection constitution based on the identified procedure; and
to configure the task generation constitution or the task selection constitution the control system is configured to:
identify a set of procedure rules corresponding to the identified procedure; and
add the identified set of rules to the constitution.

19. A method for performing automated tasks via a computer-assisted system comprising one or more repositionable structures configured to support respective instruments, and a control system operatively coupled to the one or more repositionable structures, the method comprising:
receiving a plurality of data streams from one or more data sources;
detecting a user input indicative of operation of the repositionable structures;
configuring, based on the user input, at least one of (i) which data streams are included in the plurality of data streams, (ii) a task generation constitution configured to modify how a task generation machine learning model generates tasks, and (iii) an input into a robotics transformer model configured to generate outputs used to control of the one or more repositionable structures or instruments;
analyzing, via the task generation machine learning model, the data streams to identify a plurality of tasks to be performed by the one or more repositionable structures, wherein the task generation constitution is input into the task generation machine learning model to control how the task generation machine learning model analyzes the data streams;
filtering, via a task selection machine learning model, the identified tasks, wherein a task selection constitution is input into the task selection machine learning model to control how the task selection machine learning model filters the identified tasks;
selecting a task to be performed based on an output of the task selection machine learning model; and
controlling the repositionable structures to perform the selected task.

20. One or more non-transitory, computer-readable media storing instructions that, when executed by a control system of a computer-assisted system comprising one or more repositionable structures configured to support respective instruments, causes the control system to:
receive a plurality of data streams from one or more data sources;
detect a user input indicative of operation of the repositionable structures;
configure, based on the user input, at least one of (i) which data streams are included in the plurality of data streams, (ii) a task generation constitution configured to modify how a task generation machine learning model generates tasks, and (iii) an input into a robotics transformer model configured to generate outputs used to control of the one or more repositionable structures or instruments;
analyze, via the task generation machine learning model, the data streams to identify a plurality of tasks to be performed by the one or more repositionable structures, wherein the task generation constitution is input into the task generation machine learning model to control how the task generation machine learning model analyzes the data streams;
filter, via a task selection machine learning model, the identified tasks, wherein a task selection constitution is input into the task selection machine learning model to control how the task selection machine learning model filters the identified tasks;
select a task to be performed based on an output of the task selection machine learning model; and
control the repositionable structures to perform the selected task.
